(19) United States
(12) Patent Application Publication
(10) Pub. No.: US 2007/0207992 A1
(43) Pub. Date: Sep. 6, 2007

(54) Geldanamycin derivatives and method of use thereof

(75) Inventors: David Wenkert, Okemos, MI (US); Leslie Kuhn, Haslett, MI (US); Erica Scheller, Stockbridge, MI (US); Michael Kron, Mequon, WI (US); Yuehai Shen, Brighton, MI (US)

Correspondence Address: Ian C. McLeod;IAN C. MCLEOD, P.C., 2190 Commons Parkway, Okemos, MI, 48864, US

(73) Assignee: Board of Trustees of Michigan State University, East Lansing, MI (US)

(21) Appl. No.: 11/708,226

(22) Filed: Feb. 20, 2007

Related U.S. Application Data
(60) Provisional application No. 60/775,170, filed on Feb. 21, 2006.

(51) Int. Cl. A61K 31/397; C07D 403/02

(52) U.S. Cl. 514/183; 540/461; 514/210.21

(57) ABSTRACT

The present invention relates to novel geldanamycin derivatives which have antitumor and antiparasitic properties. The geldanamycin derivatives disclosed herein have antitumor properties in humans due to their interaction with human heat shock protein 90 (hsp90). The human parasites Plasmodium falciparum, Trypanosoma Cruzi, and Leishmania donovani are lethally susceptible to exposure to geldanamycin via complexation of geldanamycin with their homologs (Pfhsp90, hsp83, and hsp90, respectively) of the human hsp90. The geldanamycin derivatives disclosed herein also interact with these parasitic hsp90 homologs so as to have antiparasitic properties.

### CROSS-REFERENCE TO RELATED APPLICATIONS

This application claims benefit to U.S. Provisional Application Ser. No. 60/775,170, filed Feb. 21, 2006, which is incorporated herein by reference in its entirety.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to novel geldanamycin derivatives which have antitumor and antiparasitic properties.

(2) Description of the Related Art

U.S. Pat. No. 6,872,715 to Santi et al. discloses benzoquinone ansamycin analogs for the treatment of cancer and other diseases or conditions characterized by undesired cellular proliferation or hyperproliferation. Therapies involving the administration of such benzoquinone ansamycin analogs, optionally in combination with an inhibitor of an Hsp90 interacting protein, are disclosed to treat cancer and non-cancerous disease conditions.

U.S. Pat. Nos. 6,855,705, 6,870,049, 6,875,863, and 6,887,993 to Tian et al. disclose 11-O-methylgeldanamycin derivatives as anti-proliferative agents. The 11-O-metylgeldanamycin derivatives have various groups on the carbon at position 17 and the nitrogen at position 22. There are no derivatives having modifications at position 7.

U.S. Patent Application Publication No. 2004/0053909 Al to Snader et al. discloses a geldanamycin derivative exhibiting preliminary in vivo activity, including oral in vivo activity, and a method of treating or preventing cancer in a host comprising administering a geldanamycin derivative to a host in an amount sufficient to treat or prevent cancer.

Various geldanamycin derivatives have been described as potential anticancer agents and as specific inhibitors of heat shock protein 90 (Hsp90). Kumar et al., The heat shock protein 90 of Plasmodium falciparum and antimalarial activity of its inhibitor, geldanamycin, Malaria Journal 2:30 (2003) have hypothesized that since Plasmodium falciparum is reported to have a homolog of Hsp90, geldanamycin could inhibit this molecule and therefore have antiparasitic properties. Kumar et al. teaches that Plasmodium falciparum growth in human erythrocyte culture is inhibited by geldanamycin with an IC₅₀ of 20 nM. However, the natural compound geldanamycin is too toxic for therapeutic use.

The related art describe geldanamycin derivatives which may be useful as anticancer agents and that geldanamycin has antiparasitic properties. However, there still exists a need for new geldanamycin derivatives having antiparasitic activity, and preferably having minimal human toxicity.

### OBJECTS

Therefore, it is an object of the present invention to provide novel geldanamycin derivatives which have antitumor and antiparasitic properties.

These and other objects will become increasingly apparent by reference to the following description.

### SUMMARY OF THE INVENTION

The present invention provides a geldanamycin derivative with the structure: 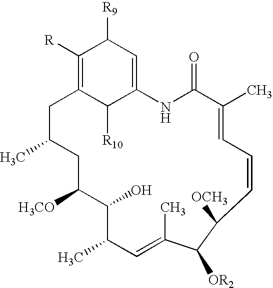
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R² is an aminoacyl group with 1 to 6 carbon atoms, an acyl group with a phenyl moiety (i.e., a benzoyl group), or an acyl group with an alkyl or cycloalkyl moiety comprising 3 to 4 carbon atoms; and wherein R₉ and R₁₀ are either hydroxy groups or keto groups and amine or amino salts thereof.

In further embodiments, R² is a propionyl, n-butyryl, α-methylpropionyl, benzoyl, or cyclopropylcarboxyl group. In further embodiments, R is an azetidinyl moiety. In still further embodiments, the derivative is 17-(1-azetidinyl)-7-butyryl-7-decarbamyl-17-demethoxygeldanamycin, 17-(1-azetidinyl)-7-(cyclopropanyl)-carbonyl-7-decarbamyl-17-demethoxygeldanamycin, 17-(1-azetidinyl)-7-benzoyl-7-decarbamyl-17-demethoxygeldanamycin, 17-(1-azetidinyl)-7-decarbamyl-17-demethoxy-7-propionyl-geldanamycin, or 17-(1-azetidinyl)-7-decarbamyl-17-demethoxy-7-isobutyryl-geldanamycin and hydroquinone derivatives thereof.

The present invention provides a geldanamycin derivative with the structure: 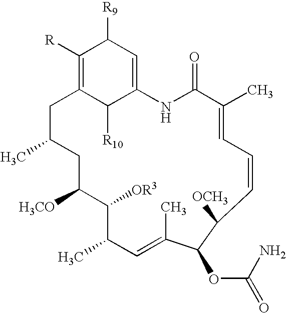
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R³ is an acyl group with 2 to 8 carbon atoms and one or more positively charged groups; and wherein R₉ and R₁₀ are either hydroxy groups or keto groups and amine or amino salts thereof. In further embodiments, the positively charged group comprises nitrogen. In further embodiments, R³ is an γ-ammonium butyryl acyl group, an α-(2-ammoniumethyl)-γ-ammonium butyryl acyl group, or a 4-(Boc)aminobutyryl acyl group. In further embodiments, R is an azetidinyl moiety. In still further embodiments, the derivative is 11-O-(4-ammoniumbutyryl)-17-(1-azetidinyl)-17-demethoxygeldanamycin trifluoroacetate. In regard to the latter, the anion need not be trifluoroacetate. Other anions include acetate, halide ions (including fluoride, chloride, bromide, and iodide), benzoate, phenylsulfonate, hydrogen sulfate, and dihydrogen phosphate. Thus, in still further embodiments, the derivative is an 11-O-(4-ammoniumbutyryl)-17-(1-azetidinyl)-17-demethoxygeldanamycin salt having an acetate, fluoride, chloride, bromide, iodide, benzoate, phenylsulfonate, hydrogen sulfate or dihydrogen sulfate anion as a counterion. It is to be understood that the counterion is not limited to these anions and that a person of skill in the art would recognize other anions that could be used. Therefore, any other anions that can be used as counterions are encompassed by the present invention.

The present invention provides a geldanamycin derivative having one or more substitutions, R, R², and R³, with the structure: 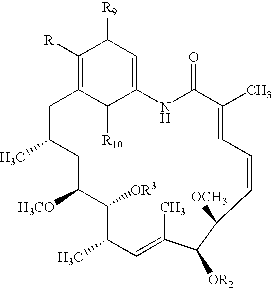
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R² and R³ are α-methylpropionyl groups; and wherein R₉ and R₁₀ are either hydroxy groups or keto groups and amine or amino salts thereof. In further embodiments, the derivative is 17-(1-azetidinyl)-7-decarbamyl-7,11-diisobutyryl-17-demethoxygeldanamycin.

The present invention provides a geldanamycin derivative having one or more substitutions, R, R¹, R², R³ and R⁴, with the structure: 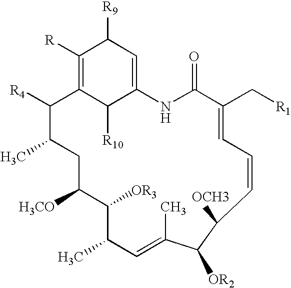
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and wherein the one or more substitutions are organic groups selected from the groups consisting of: attached to the methylene carbon atom at position 2, R¹ is an organic group with 1 to 2 carbon atoms and a hydrogen-bonding atom replacing the hydrogen; at position 7, R² is an aminoacyl group with 1 to 6 carbon atoms, an acyl group with an alkyl moiety containing 3 to 4 carbon atoms, an acyl group with a phenyl moiety (i.e. a benzoyl group), or an acyl group with a cycloalkyl moiety containing 3 to 4 carbon atoms replacing the carbamoyl group; at position 11, R³ is an acyl group with 2 to 8 carbon atoms and one or more positively charged groups replacing the hydroxyl; at position 15, R⁴ is a hydrogen-bonding atom replacing the hydrogen; and wherein R₉ and R₁₀ and are either hydroxy groups or keto groups and amine or amino salts thereof. In further embodiments, R is an azetidinyl moiety.

The present invention provides a geldanamycin derivative with the structure: 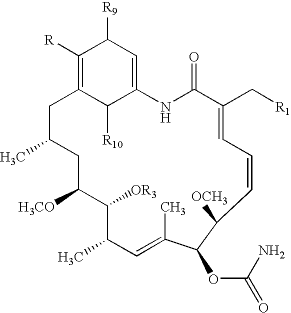
wherein R is methoxy or an R⁵R⁶N amine, where R⁵ and R⁶ are independently H, C₁-C₈ alkyl, Cₗ-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R¹ is an organic group with 1 to 2 carbon atoms and with a hydrogen-bonding atom; and wherein R₉ and R₁₀ and are either hydroxy groups or keto groups and amine or amino salts thereof. In further embodiments, the hydrogen-bonding atom is an oxygen or a nitrogen atom. In still further embodiments R¹ has a hydroxyl, ether, primary amine, secondary amine, or tertiary amine group and hydroquinone derivatives thereof.

The present invention provides a geldanamycin derivative with the structure: 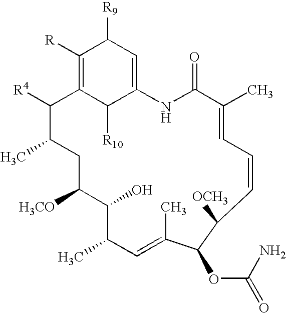
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R⁴ comprises a hydrogen-bonding atom which is bonded to the carbon atom; and wherein R₉ and R₁₀ and are either hydroxy groups or keto groups and amine or amino salts thereof. In further embodiments, the hydrogen-bonding atom is an oxygen or a nitrogen. In further embodiments, R⁴ is a hydroxyl, alkoxy, primary amine, secondary amine, tertiary amine group, primary ammonium, secondary ammonium, or tertiary ammonium group.

The present invention provides a method of inhibiting Plasmodium falciparum comprising providing a geldanamycin derivative having one or more substitutions, R¹, R², R³ and R⁴, with the structure: 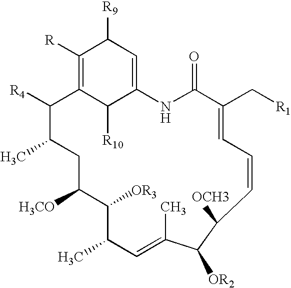
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and wherein the one or more substitutions are organic groups selected from the groups consisting of: attached to the methylene carbon atom at position 2, R¹ is an organic group with 1 to 2 carbon atoms and a hydrogen-bonding atom replacing the hydrogen; at position 7, R² is an aminoacyl group with 1 to 6 carbon atoms, an acyl group with an alkyl moiety containing 3 to 4 carbon atoms, an acyl group with a phenyl moiety (i.e. a benzoyl group), or an acyl group with a cycloalkyl moiety containing 3 to 4 carbon atoms replacing the carbamoyl group; at position 11, R³ is an acyl group with 2 to 8 carbon atoms and one or more positively charged groups replacing the hydroxyl; at position 15, R⁴ is a hydrogen-bonding atom replacing the hydrogen and wherein R₉ and R₁₀ are either hydroxy groups or keto groups and amine or amino salts thereof; so as to inhibit the Plasmodium falciparum.

In further embodiments of the method, R is an azetidinyl moiety. In further embodiments, R2 is a propionyl, n-butyryl, α-methylpropionyl, benzoyl, or cyclopropylcarboxyl group. In still further embodiments, the derivative is 17-(1-azetidinyl)-7-butyryl-7-decarbamyl-17-demethoxygeldanamycin, 17-(1-azetidinyl)-7-(cyclopropanyl)-carbonyl-7-decarbamyl-17-demethoxygeldanamycin, 17-(1-azetidinyl)-7-benzoyl-7-decarbamyl-17-demethoxygeldanamycin, 17-(1-azetidinyl)-7-decarbamyl-17-demethoxy-7-propionyl-geldanamycin, or 17-(1-azetidinyl)-7-decarbamyl-17-demethoxy-7-isobutyryl-geldanamycin and hydroquinone derivatives thereof. In still further embodiments, the positively charged group comprises nitrogen. In still further embodiments, R3 is a γ-ammonium butyryl acyl group or an α-(2-ammoniumethyl)-γ-ammonium butyryl acyl group. In still further embodiments, the derivative is 11-O-(4-ammoniumbutyryl)-17-(1-azetidinyl)-17-demethoxygeldanamycin trifluoroacetate. In regard to the latter, the anion need not be trifluoroacetate. Other anions include acetate, halide ions (including fluoride, chloride, bromide, and iodide), benzoate, phenylsulfonate, hydrogen sulfate, and dihydrogen phosphate.

The present invention provides a method of inhibiting a parasite heat shock protein homolog of human heat shock protein 90 (hsp90) comprising providing a geldanamycin derivative having one or more substitutions, R¹, R², R³ and R⁴, with the structure: 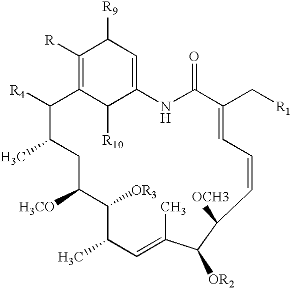
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and wherein the one or more substitutions are organic groups selected from the groups consisting of: attached to the methylene carbon at position 2, R¹ is an organic group with 1 to 2 carbon atoms and a hydrogen-bonding atom replacing the hydrogen; at position 7, R² is an aminoacyl group with 1 to 6 carbon atoms, an acyl group with an alkyl moiety containing 3 to 4 carbon atoms, an acyl group with a phenyl moiety (i.e. a benzoyl group), or an acyl group with a cycloalkyl moiety containing 3 to 4 carbon atoms replacing the carbamoyl group; at position 11, R³ is an acyl group with 2 to 8 carbon atoms and one or more positively charged groups replacing the hydroxyl; at position 15, R⁴ is a hydrogen-bonding atom replacing the hydrogen; and wherein R₉ and R₁₀ and are either hydroxy groups or keto groups and amine or amino salts thereof so as to inhibit the parasitic heat shock proteins. In further embodiments, the parasite is selected from the group consisting of Plasmodium falciparum, Trypanosoma cruzi, and Leishmania donovani. In still further embodiments, the parasite is selected from the group consisting of protozoan, nematode, cestode and trematode parasites.

The present invention provides a method of treating a patient with a parasitic disease comprising providing to the patient a geldanamycin derivative having one or more substitutions, R¹, R², R³ and R⁴, with the structure: 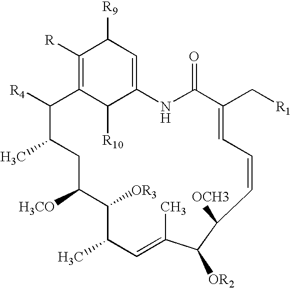
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and wherein the one or more substitutions are organic groups selected from the groups consisting of: attached to the methylene carbon atom at position 2, R¹ is an organic group with 1 to 2 carbon atoms and a hydrogen-bonding atom replacing the hydrogen; at position 7, R² is an aminoacyl group with 1 to 6 carbon atoms, an acyl group with an alkyl moiety containing 3 to 4 carbon atoms, an acyl group with a phenyl moiety (i.e. a benzoyl group), or an acyl group with a cycloalkyl moiety containing 3 to 4 carbon atoms replacing the carbamoyl group; at position 11, R³ is an acyl group with 2 to 8 carbon atoms and one or more positively charged groups replacing the hydroxyl; at position 15, R⁴ is a hydrogen-bonding atom replacing the hydrogen; and wherein R₉ and R₁₀ are either hydroxy groups or keto groups and amine or amino salts thereof to inhibit a parasite heat shock protein homolog of human heat shock protein 90 (hsp90), so as to treat the disease.

In further embodiments, the parasite is selected from the group consisting of Plasmodium falciparum, Trypanosoma cruzi, and Leishmania donovani. In still further embodiments, the parasite is selected from the group consisting of protozoan, nematode, cestode and trematode parasites.

The present invention provides a method of treating a patient with cancer comprising providing to the patient a geldanamycin derivative having one or more substitutions, R, R², and R³, with the structure: 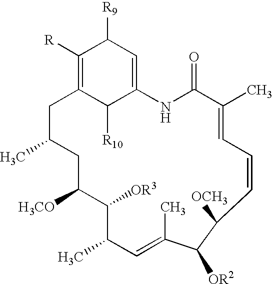
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R² is an aminoacyl group with 1 to 6 carbon atoms, an acyl group with a phenyl moiety, or an acyl group with an alkyl or cycloalkyl moiety comprising 3 to 4 carbon atoms; wherein R³ is an acyl group with 2 to 8 carbon atoms and one or more positively charged groups; and wherein R₉ and R₁₀ are either hydroxy groups or keto groups and amine or amino salts thereof so as to treat the patient with cancer.

In further embodiments, R² is a propionyl, n-butyryl, α-methylpropionyl, benzoyl, or cyclopropylcarboxyl group. In further embodiments, R³ is a γ-ammonium butyryl acyl group or an α-(2-ammoniumethyl)-γ-ammonium butyryl acyl group. In further embodiments, R is an azetidinyl moiety.

### BRIEF DESCRIPTION OF THE DRAWINGS

FIG. 1 shows the structure of geldanamycin with numbering scheme of ring carbon atoms shown.

FIG. 2 shows a synthetic pathway for geldanamycin R¹ derivatives of the present invention.

FIG. 3 shows a synthetic pathway for geldanamycin R⁴ derivatives of the present invention.

FIG. 4 shows the chemical structure of the dihydroform of geldanamycin, which is dihydrogeldanamycin.

FIG. 5 shows the general structural formula of the dihydrogeldanamycin derivative analogs of the compounds set forth in the Summary of the Invention defining R and R² at C7 and C17 (see FIG. 1) for the position numbering.

FIG. 6 is the structural formula of the dihydrogeldanamycin derivative analogs of the compounds set forth in the Summary of the Invention defining R at C17 as a variable.

FIG. 7 is a structural formula of the dihydrogeldanamycin derivative analogs including R at C17, RR² at C7 and R³ at C11.

FIG. 8 is a structural formula of dihydrogeldanamycin derivative analogs of the compounds set forth in the Summary of the Invention defining R at C17, R¹ at C2, R³ at C11, R⁴ at C15 as variables.

FIG. 9 is a structural formula of dihydrogeldanamycin derivative analogs of the compounds set forth in the Summary of the Invention defining R at C17 and R at C2 as variables.

FIG. 10 is a structural formula of dihydrogeldanamycin derivative analogs of the compounds set forth in the Summary of the Invention defining R and C17 and R⁴ at C15 as variables.

FIG. 11 is a dihydrogeldanamycin derivative of the 17-(1-Azetidinyl)-17-demethoxygeldanamycin (Compound G) of Example 2.

FIG. 12 is a dihydrogeldanamycin derivative of the 17-(1-Azetidinyl)-7-decarbamyl-17-demethoxygeldanamycin (Compound D) of Example 2.

FIG. 13 is a dihydrogeldanamycin derivative of the 17-(1-Azetidinyl)-7-decarbamyl-17-demethoxy-7-propionylgeldanamycin (Compound F) of Example 2.

FIG. 14 is a dihydrogeldanamycin derivative of the 17-(1-Azetidinyl)-7-butyryl-7-decarbamyl-17-demethoxygeldanamycin (Compound B) of Example 2.

FIG. 15 is a dihydrogeldanamycin derivative of the 17-(1-Azetidinyl)-7-decarbamyl-17-demethoxy-7-isobutyryl-geldanamycin.

FIG. 16 is a dihydrogeldanamycin derivative of the 17-(1-Azetidinyl)-7-decarbamyl-7,11-diisobutyryl-17-demethoxygeldanamycin.

FIG. 17 is a dihydrogeldanamycin derivative of the 17-(1-Azetidinyl)-7-(cyclopropanyl)carbonyl-7-decarbamyl-17-demethoxygeldanamycin (Compound C).

FIG. 18 is a dihydrogeldanamycin derivative of the 17-(1-Azetidinyl)-7-benzoyl-7-decarbamyl-17-demethoxygeldanamycin (Compound E).

FIG. 19 is a dihydrogeldanamycin derivative of the 17-(1-Azetidinyl)-11-O-[4-(Boc)Aminobutyryl]-17-demethoxygeldanamycin.

FIG. 20 is a dihydrogeldanamycin derivative of the 11-O-(4-Aminobutyryl)-17-(1-azetidinyl)-17-demethoxygeldanamycin trifluoroacetate (Compound A).

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “alkyl” as used herein refers to an optionally substituted, straight or branched chain hydrocarbon moiety having the specified number of carbon atoms in the chain. When the number of carbon atoms is otherwise not specified there are up to 8 carbon atoms in the chain.

The term “alkenyl” as used herein refers to an optionally substituted, straight or branched chain hydrocarbon moiety having at least one carbon-carbon double bond and having the specified number of carbon atoms in the chain. When the number of carbon atoms is otherwise not specified there are up to 8 carbon atoms in the chain.

The term “alkynyl” as used herein refers to an optionally substituted, straight or branched chain hydrocarbon moiety having at least one carbon-carbon triple bond and having the specified number of carbon atoms in the chain. When the number of carbon atoms is otherwise not specified there are up to 8 carbon atoms in the chain.

The term “aryl” as used herein refers to a monocyclic or bicyclic aromatic hydrocarbon ring system having 6 to 12 carbon atoms in the ring portion, such as phenyl, napthyl, and biphenyl moieties, each of which is optionally substituted at one or more positions.

The term “alkylaryl” as used herein refers to an aryl bonded directly to an alkyl moiety, including but not limited to benzyl and phenylethyl.

The term “cycloalkyl” as used herein refers to an optionally substituted, saturated cyclic hydrocarbon ring system, preferably containing 1 to 3 rings and 3 to 7 carbons per ring which may be further fused with an unsaturated C₃-C₇ carbocyclic ring. Cycloalkyl ring systems include, but are not limited to cyclopropyl, cyclobutyl, cyclopentyl, cyclohexyl, cycloheptyl, cyclooctyl, cyclodecyl, cyclododecyl, and adamantyl.

The term “heterocyclic” or “heterocyclo” as used herein refers to an optionally substituted, fully saturated or unsaturated, aromatic or nonaromatic ring system, for example, which is a 4 to 7 membered monocyclic, 7 to 11 membered bicyclic, or 10 to 15 membered tricyclic ring system, which has at least one heteroatom in at least one carbon atom-containing ring. The heteroatoms can be selected from N, O and S.

The term “heteroaryl” as used herein refers to a heterocycle in which the ring system is aryl. Each ring of the heterocyclic group containing a heteroatom can have 1, 2 or 3 heteroatoms selected from N, O and S.

Kumar et al., The heat shock protein 90 of Plasmodium falciparum and antimalarial activity of its inhibitor, geldanamycin, Malaria Journal 2:30 (2003) hereby incorporated herein by reference in its entirety, reports that geldanamycin is a promising antimalarial drug. Banumathy et al. Heat Shock Protein 90 Function Is Essential for Plasmodium falciparum Growth in Human Erythrocytes, J. Biol. Chem., vol. 278, No. 20, pp. 18336-18345, hereby incorporated herein by reference in its entirety, analyzed Plasmodium falciparum Hsp 90 (PfHsp90) and discloses a putative geldanamycin binding domain in PfHsp90.

Antiparasitic drug development: Inhibition of parasitic heat shock protein 90. The human parasites Plasmodium falciparum, Trypanosoma Cruzi, and Leishmania donovani are lethally susceptible to exposure to geldanamycin via complexation of the latter with their homologs (Pfhsp90, hsp83, and hsp90, respectively) to human heat shock protein 90 (hsp90). The compounds of the present invention were designed utilizing structure-based molecular modeling, chemical synthesis, and the resulting compounds are assessed for specific interaction by means of binding studies, as well as the determination of drug concentrations needed for killing of parasites.

Members of the heat shock protein 90 (hsp90) family are ubiquitous, abundant and essential proteins in all eukaryotic organisms. Hsp90 helps in the regulation of activity, turnover, and trafficking of various critical proteins. It facilitates in the folding and regulation of proteins in cellular signaling, such as transcription factors, steroid receptors, and protein kinases. It belongs to the structural protein family of GHKL ATPases. The function of hsp90 is blocked by the natural product geldanamycin. This natural product binds in the ATP binding pocket of the amino-terminal domain of hsp90. The specificity of this natural product for hsp90 is seen by the paucity of other known proteins to which they bind.

That hsp90 function is critical for parasitic viability is seen by the lethality of geldanamycin to a number of parasites. The human parasites Plasmodium falciparum, Trypanosoma Cruzi, Leishmania donovani are examples for which geldanamycin has such a fatal effect. In fact, it has been pointed out that the relatively low IC₅₀ (20 nM) of geldanamycin against P. falciparum contrasted with the higher serum concentrations (in the micromolar range) in humans without overt toxicity makes geldanamycin itself a potential antimalarial agent. Each of the aforementioned parasites expresses its own hsp90 homolog (Pfhsp90, hsp83, hsp90, and hsp90, respectively). These hsp90 homologs display similarity to human hsp90 in structure and function. However, differences exist between these homologs to human hsp90. For example, the hsp90 from Plasmodium falciparum shows 59% identity and 69% similarity in protein sequence to that of human hsp90. Though this parasitic hsp90 has been shown to bind geldanamycin, it does haves some differences from human hsp90 in its ATP-binding pocket.

Structure-based molecular modeling studies have been done to compare the known crystal structure of the amino-terminal domain of human hsp90 bound to geldanamycin with the structure expected for the analogous domain of Plasmodium falciparum hsp90 bound to geldanamycin. These studies indicate the following amino acid changes (i.e., human to P. falciparum) in the geldanamycin binding site: Ser52→Ala52, Val91→Ile91, Ala101→Asn101, Cys112→Arg112, Thr149→Val149, Thr152→Ser152, His154→Asn154, and Val186→Ile186. Of these changes, those of positions 52, 112, 154, and 186 represent ones of which advantage can be taken for increasing the binding affinity of geldanamycin derivatives for Plasmodium falciparum hsp90 over human hsp90 as described below.

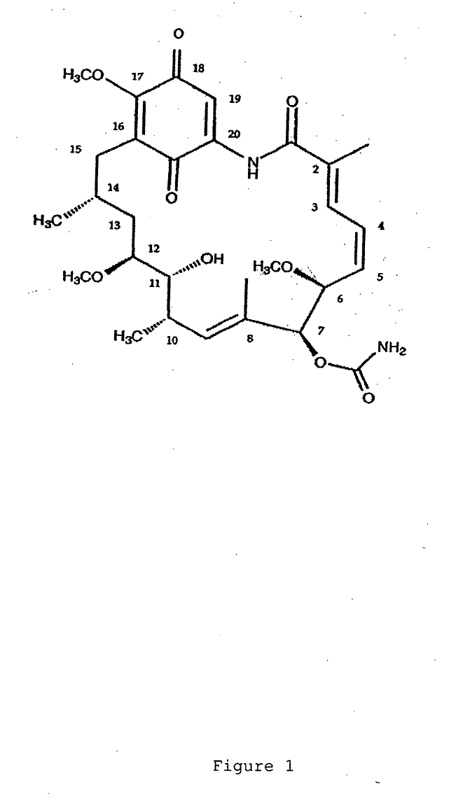

The structure of geldanamycin has been described by Sasaki et al. in the Journal of the American Chemical Society 92:26 pp. 7591-7593 (Dec. 30, 1970). FIG. 1 illustrates the numbering scheme of geldanamycin. Geldanamycin derivatives that can help discriminate between these two hsp90's are derivatized at the methyl group on ring carbon 2, the carbamyl group on ring carbon 7, the hydroxyl group of ring carbon 11, the methoxy group on ring carbon 12, and the ring carbon atom of position 15. A substituent on the methyl group of ring carbon 2 that can act as a hydrogen-bond acceptor increases positive interaction with the Arg112 present in the P. falciparum hsp90. Specifically, a hydroxyl group or a dimethylamino group are appropriate substituents to effect such an interaction. It is possible that this interaction can be further enhanced by placement of a methylene carbon between such substituents and the methyl group to facilitate this interaction. In regard to the carbamyl group on ring carbon 7 of geldanamycin, if the group is removed and the resulting free hydroxyl group on ring carbon 7 is esterified with an acyl group of three or four carbon atoms, the hydrophobic pocket created by the Ser52→Ala52 change would be filled. Such acyl substituents can be, for example, propionyl, n-butyryl, iso-butyryl and cyclopropylcarboxyl groups. For the hydroxyl group on ring carbon 11, the placement of an acyl group that has an ε-amino group(s) can facilitate creating a salt bridge to Asp102 and Glu62 of P. falciparum hsp90 and repel His154 of human hsp90. Such acyl groups can be, for example, the protonated ammonium salts of 4-aminobutyryl and 4-amino-2-(2-aminoethyl)butyryl groups. For the methoxy group of ring carbon 12, if this group is exchanged with another alkoxy group that was longer by one or two carbon atoms, i.e., ethoxy or propoxy groups, the hydrophobic interaction interactions can be enhanced with Ile186. In regard to ring carbon 15, placement of an α-hydroxyl group on this carbon facilitates hydrogen bonding with Arg112 present in P. falciparum hsp90. Finally, the combination of any or all of these changes can also result in a more potent and/or discriminatory antiparasitic drug than that seen with only one such change.

Chemical Synthesis: For the making of such changes in geldanamycin, synthetic strategies must be considered. Geldanamycin synthetic approaches are described by Andrus et al. “Total Synthesis of (+)-Geldanamycin and (−)-o-Quinogeldanamycin: Asymmetric Glycolate Aldol Reactions and Biological Evaluation” J. Org. Chem. 68 pp 8162-8169 (2003); Schnur et al. “Inhibition of the Oncogene Product p185^{erbB-2} in Vitro and in Vivo by Geldanamycin and Dihydrogeldanamycin Derivatives” J. Med. Chem. 38 pp. 3806-3812 (1995); Schnur et al. “erbB-2 Oncogene Inhibition by Geldanamycin Derivatives: Synthesis, Mechanism of Action, and Structure-Activity Relationships” J. Med. Chem. 38 pp. 3813-3820 (1995); and Schnur and Corman J. “Tandem [3,3]-Sigmatropic Rearrangements in an Ansamycin: Stereospecific Conversion of an (S)-Allylic Alcohol to an (S)-Allylic Amine Derivative” J. Org. Chem. 59 pp. 2581-2584 (1994) each of which are hereby incorporated herein by reference in its entirety. Several geldanamycin derivatives have been made from naturally-occurring geldanamycin itself. Some of this chemistry can be utilized in making the geldanamycin derivatives herein. Specifically, naturally-occurring geldanamycin can be used for making the derivatives with changes at the carbon 7 and carbon 11 sites. 17-Dialkylamino-, 17-monoalkylamino-, and 17-amino-17-demethoxygeldanamycin derivatives have been found to bind human hsp90 effectively. These compounds have the 17-amino substituent pointing out of the geldanamycin binding pocket of hsp90 and are expected to also bind P. falciparum hsp90 effectively. These derivatives have been found to be effectively decarbamylated upon treatment with strong base (e.g., potassium tert-butoxide). This treatment results in a free hydroxyl group being present at ring carbon 7. Although the decarbamylated geldanamycin derivatives have both free 7- and 11-hydroxyl groups, and although the 11-hydroxyl group of geldanamycin and derivatives can be esterified, it has been shown that for decarbamylated derivatives the 7-hydroxyl group can be selectively esterified. Thus, we have found the decarbamylated geldanamycin compounds can be esterified with an appropriate acyl chloride (propionyl, n-butyryl, iso-butyryl and cyclopropylcarboxyl chloride) in the presence of base (e.g., 4-dimethylaminopyridine) to give the previously described desired 7-postioned esters. When 7-ester substituents are desired on geldanamycin itself, rather than on the 17-amino-17-demethoxygeldanamycin derivatives, this same decarbamylation procedure with the strong, nonnucleophilic base such as tert-butoxide can give a decarbamylated geldanamycin which can be esterified in like manner.

Tian et al. “Synthesis and biological activities of novel 17-aminogeldanamycin derivatives”, Bioorg. Med. Chem. 12 (2004) pp. 5317-5329, U.S. Pat. Nos. 6,855,705, 6,870,049, 6,875,863, and 6,887,993 to Tian et al. and U.S. Pat. No. 6,872,715 to Santi et al. each of which are hereby incorporated herein by reference in their entirety, describe various 17-aminogeldanamycin derivatives. Tian et al. (Bioorg. Med. Chem. 12 (2004) pp. 5317-5329) systematically investigated various C-17 side chains in 17-amino-17-demethoxygeldanamycin derivatives. Three types of side chains for the 17-amino groups were studied: side chains of a homologous series to study the effect of size; amines with hydrophilic side chains; and side chains with specific structural features including enhanced rigidity. The derivatives which were analyzed for properties such as human cancer cell cytotoxicity, human hsp90 binding activity, and water solubility. The human breast cancer cell line SKBr3 was used to study the effect of the derivatives on cancer cell growth inhibition. A number of analogs showed cancer cell growth inhibition potencies similar to that of 17-N-allylamino-17-demethoxygeldanamycin (17-AAG), but also having improved water solubility. In some embodiments, the 17-amino group on the geldanamycin derivatives of the present invention have side chains as disclosed in these references.

For the synthesis of the desired 11-position esters of the present invention, esterification of the 11-hydroxyl group is to be done with a N-protected 4-aminobutyric acid and a N,N′-protected 4-amino-2-(2-aminoethyl)butyric acid. It is desired that the amino-protecting group be such that it can be removed under mild conditions so that further potential reactions (e.g., transamination with the ring amide function) of the resulting aminoester of geldanamycin can be avoided. Also, as the 17-methoxy group of geldanamycin is easily displaced by primary amines and as a primary amine is generated upon deprotection of the aminoester, it is desired to make such 11-position esters from 17-amino-17-demethoxygeldanamycin derivatives. With these considerations in mind, it is desirable to use the triisopropylsilyloxycarbonyl (“Tsoc”) protecting group. This group can be easily be placed on primary amines by reaction of the latter with carbon dioxide in the presence of triethylamine followed by triisopropylsilyl trifluoromethanesulfonate. The group can later be removed by treatment with fluoride anion. We have made the amine “Tsoc” protected derivative of 4-aminobutyric acid, by using this protection reaction procedure on benzyl 4-aminobutyric acid followed by debenzylation with hydrogen and palladium. The “Tsoc” protected derivative of 4-aminobutyric acid is to then be esterified with 17-amino-17-demethoxygeldanamycin, 17-alkylamino-17-demethoxygeldanamycin, 17-dialkylamino-17-demethoxygeldanamycin, or 17-cycloalkylamino-17-demethoxy-geldanamycin in the presence of, e.g., dicyclohexyl-carbodiimide. A similar procedure can be followed for the placement of the 4-amino-2-(2-aminoethyl)butyryl group on the 11-hydroxyl group of 17-amino-17-demethoxygeldanamycin, 17-alkylamino-17-demethoxy-geldanamycin, 17-dialkylamino-17-demethoxygeldanamycin, or 17-cycloalkylamino-17-demethoxy-geldanamycin derivatives. Although the use of Tsoc is one possible means of protection, one preferred method for protection and deprotection utilizes the Boc protecting group as described in the Examples.

For example, a geldanamycin derivative with the 11-hydroxyl group esterified with a γ-ammoniumbutyryl group can be synthesized. The last two steps of the synthesis of this compound is the reaction of geldanamycin with 4-(N-triisopropylsilyloxycarbonylamino) -butyric anhydride and base (e.g., para-dimethylaminopyridine) to provide 11-[4-(-N triisopropylsilyloxycarbonylamino)butyryl]-geldanamycin. The latter is reacted with fluoride anion to provide the product 11-(4-aminobutyryl)-geldanamycin. In aqueous medium at neutral or lower pH, the latter exists as the charged 11-(4-ammoniumbutyryl)-geldanamycin. For the synthesis of 4-(N-triisopropylsilyloxycarbonylamino)-butyric anhydride, benzyl 4-aminobutyrate has been reacted with carbon dioxide, triisopropylsilyl trifluoromethanesulfonate and triethylamine to provide benzyl 4-(N-triisopropylsilyloxycarbonylamino)butyrate. The latter is to have the benzyl group removed by hydrogenation in the presence of catalyst (e.g., palladium on charcoal), and the 4-(N-triisopropyl-silyloxycarbonylamino)butyric acid product treated with dicyclohexylcarbodiimide to provide desired 4-(N-triisopropylsilyloxycarbonylamino)-butyric anhydride product.

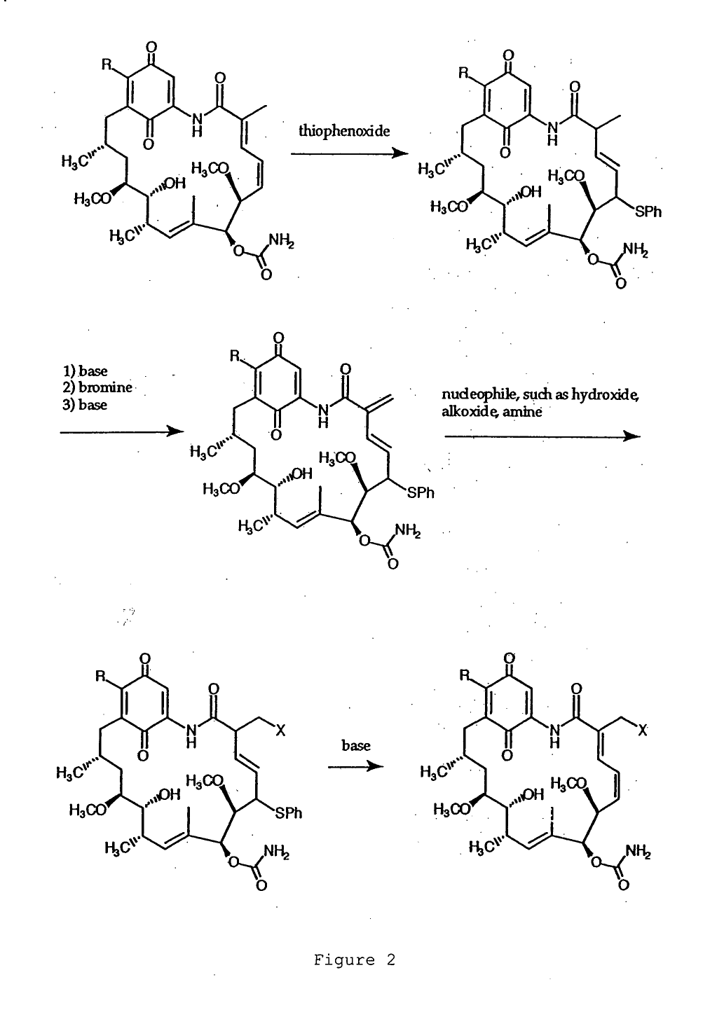

The geldanamycin derivatives with the structure: 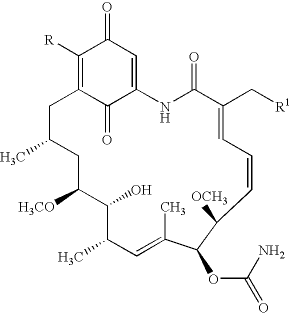
can be synthesized as shown in FIG. 2 and as described as follows. A 17-substituted-17-demethoxygeldanamycin derivative, where the 17-substituent is an amino, alkylamino, dialkylamino, or cycloalkylamino group, is used as the starting material. This is reacted with thiophenoxide anion (aprotic solvent such as tetrahydrofuran; temperature of reaction to be 0° C. to room temperature) to place a thiophenoxy group on the 5-position. Reaction with one equivalent of base such as lithium diisopropylamine (aprotic solvent such as tetrahydrofuran; temperature of reaction to be −78° C. to 0° C.), followed by one equivalent of bromine (aprotic solvent such as tetrahydrofuran; temperature of reaction to be −78° C. to 0° C.), and then one equivalent of base such as lithium diisopropylamine (aprotic solvent such as tetrahydrofuran; temperature of reaction to be −78° C. to 0° C.) will give the compound shown in the center of FIG. 2. Treatment of the latter with a nucleophile such as hydroxide anion, alkoxide anion, or an amine (aprotic solvent such as tetrahydrofuran; temperature of reaction to be −78° C. to room temperature) will put the nucleophile on the geldanamycin methyl carbon attached to position 2. Treatment with base, such as lithium diisopropylamine, (aprotic solvent such as tetrahydrofuran; temperature of reaction to be −78° C. to 20° C.) will result in loss of the thiophenoxy substituent from position 5 and give the desired derivative. Some examples are derivatives having an R¹ of —OH, —OR, —NH2, —NHR, NR′R″, and N⁺R′R″R′″.

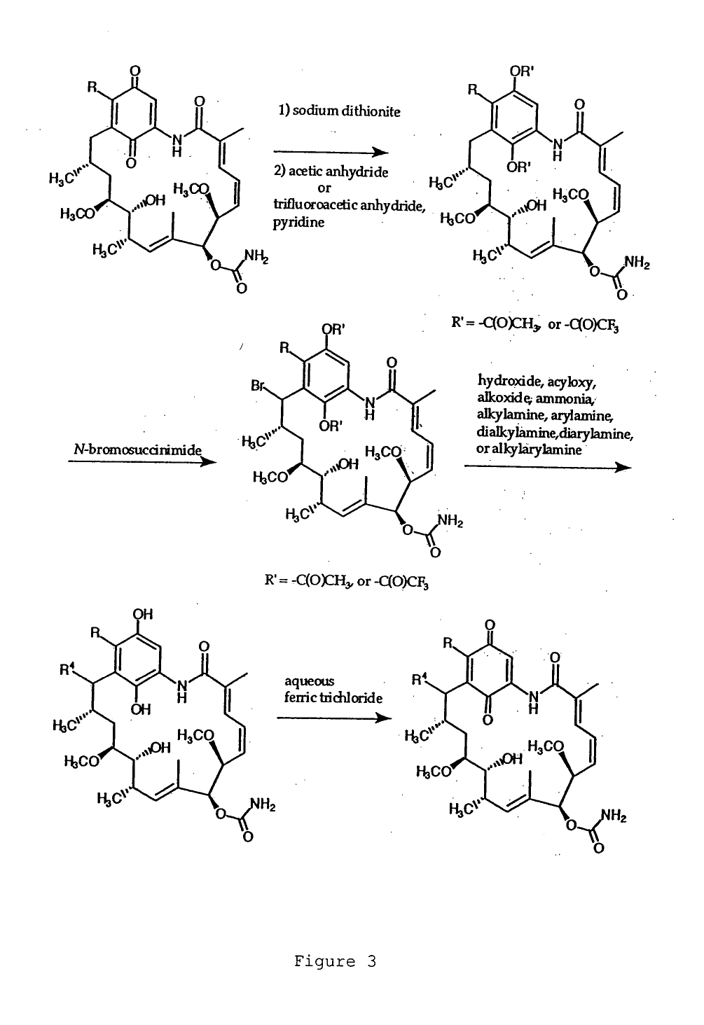

A geldanamycin derivative with the structure: 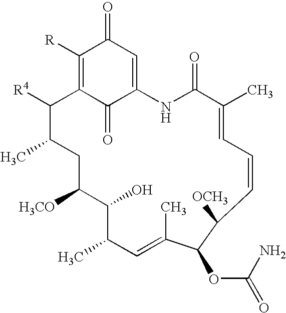
can be synthesized as shown in FIG. 3 and as described as follows. Geldanamycin or a 17-substituted-17-demethoxygeldanamycin derivative, where the 17-substituent is an amino, alkylamino, dialkylamino, or cycloalkylamino group, is used as the starting material. The benzoquinone is reduced with sodium dithionite to give dihydrogeldanamycin or 17-substituted-17-demethoxydihydrogeldanamycin as has been described in the literature (Schnur et al. “Inhibition of the Oncogene Product p185^{erB-2} in Vitro and in Vivo by Geldanamycin and Dihydrogeldanamycin Derivatives” J. Med. Chem. 38 pp. 3806-3812 (1995)). The 18- and 21-OH groups of the hydroquinone ring are then acetylated with acetic anhydride in the presence of pyridine at 0° C. to room temperature or trifluoroacetylated with trifluoroacetic anhydride in the presence of pyridine at 0° C. to room temperature. This is then reacted with N-bromosuccinimide to place a bromide atom on the 15-position carbon. (Reaction conditions will be in an anhydrous solvent such as methylene chloride at temperatures from 0° C. to room temperature.) Reaction with aqueous base at 0° C. to room temperature will give the hydroquinone derivative with a 15-hydroxy group (thus R⁴=—OH) and with the 18- and 21-OH groups no longer acylated (with either acetyl or trifluoroacetyl groups). Oxidation with aqueous iron trichloride at 0° C. to room temperature will give the desired final benzoquinone derivative with a 15-hydroxyl group. Alternatively, reaction with acetoxy anion or trifluoroacetoxy anion, followed by treatment of the resulting 15-ester product with weak aqueous base at 0° C. to room temperature will provide the desired derivative with a 15-hydroxyl group and with the 18- and 21-OH groups no longer acylated (with either acetyl or trifluoroacetyl groups). Oxidation with aqueous iron trichloride at 0° C. to room temperature will give the desired final benzoquinone derivative with a 15-hydroxyl group. For synthesis of this derivative type in which R⁴ is an alkoxy derivative the above 15-bromoderivative can be treated with an alkoxide anion at 0° C. to room temperature to give the hydroquinone product (R⁴=—OR, where R is an alkyl group or an aromatic group) and with the 18- and 21-OH groups no longer acylated (with either acetyl or trifluoroacetyl groups). Oxidation with aqueous iron trichloride at 0° C. to room temperature will give the desired final benzoquinone derivative with a 15-alkoxy group. For synthesis of this derivative type in which R⁴ is an amino group (R⁴=—NH₂; thus for synthesizing a derivative that is a primary amine), an alkyl or aryl amino group (R⁴=—NHR, where R is an alkyl or aryl substituent; thus for synthesizing a derivative that is a primary secondary), or dialkyl, diaryl, or alkylaryl amino group (R⁴=—NR′R″, where R′ and/or R″ is an alkyl and/or aryl substituent; thus for synthesizing a derivative that is a primary secondary), the above 15-bromoderivative can be treated ammonia (to give a primary amine derivative), an alkyl or aryl amine (to give a secondary amine derivative), or a dialkyl, diaryl, or alkylaryl amine (to give a tertiary amine derivative) at 0° C. to room temperature to give the hydroquinone product (R⁴=—NH₂, —NHR, or —NR′R″) and with the 18- and 21-OH groups no longer acylated (with either acetyl or trifluoroacetyl groups). Oxidation with aqueous iron trichloride at 0° C. to room temperature will give the desired final benzoquinone derivative with a 15-amino-, 15-alkyl- or aryl-amino, or a 15-dialkyl-, -diaryl-, or -alkylaryl-amino group.

Parasitic Lethality: The geldanamycin derivatives were subjected to further testing for lethality to the P. falciparum parasite itself. IC₅₀ values were then obtained as illustrated in Table 1 in Example 3. Samples of the literature compound 17-N-allyl-17-demethoxygeldanamycin was synthesized and tested for killing of parasites. Preferably the drugs are selective for parasitic hsp90 homologs over human hsp90 and have minimal human toxicity. Geldanamycin derivatives of this type have potential for being novel drugs in the treatment of parasitic diseases. The medical community will be able to use these new anti-infective medications alone or in combination with other anti-infective medications. For example, the geldanamycin derivatives of the present invention can be used in combination with chloroquine, since geldanamycin and chloroquine are synergistic inhibitors of Plasmodium falciparum growth as described in Kumar et al. Malaria Journal 2:30 (2003).

The human parasites Plasmodium falciparum, Trypanosoma Cruzi, and Leishmania donovani are lethally susceptible to exposure to geldanamycin via complexation of the latter with their homologs (Pfhsp90, hsp83, and hsp90, respectively) to human heat shock protein 90 (hsp90). The present invention involves design and synthesis of geldanamycin (an ansamycin) derivatives that preferably will selectively interact with parasitic hsp90 homologs over human hsp90. This involves structure-based molecular modeling, chemical synthesis, and binding studies to assess the interaction of the molecules, as well as the determination of drug concentrations needed for killing of parasites.

Therapeutic Use: In a preferred embodiment, one or more of the geldanamycin derivatives for treating a patient with a parasitic infection, cancer or tumor are provided to the patient at an inhibitory dose, which is at an amount which does not kill normal cells in the patient, in a pharmaceutically acceptable carrier. In some embodiments, the inhibitory dose is the 50% growth inhibitory value for the geldanamycin derivatives for the particular parasitic infection, cancer or tumor afflicting the patient. As such, the geldanamycin derivatives are processed with pharmaceutical carrier substances by methods well known in the art such as by means of conventional mixing, granulating, coating, suspending and encapsulating methods, into the customary preparations for oral administration. Thus, antiparasitic, anticancer or antitumor geldanamycin derivative preparations for oral application can be obtained by combining one or more of the geldanamycin derivatives with solid pharmaceutical carriers; optionally granulating the resulting mixture; and processing the mixture or granulate, if desired and/or optionally after the addition of suitable auxiliaries, into the form of tablets or dragee cores. The geldanamycin derivatives can be given intravenously (IV) as an aqueous-dimethylsulfoxide solution. The geldanamycin derivatives can be provided intravenously as an anticancer drug.

Suitable pharmaceutical carriers for solid preparations are, in particular, fillers such as sugar, for example, lactose, saccharose, mannitol or sorbitol, cellulose preparations and/or calcium phosphates, for example, tricalcium phosphate or calcium hydrogen phosphate; also binding agents, such as starch paste, with the use, for example, of maize, wheat, rice or potato starch, gelatin, tragacanth, methyl cellulose, hydroxypropylmethyl cellulose, sodium carboxymethyl cellulose and/or polyvinylpyrrolidone, esters of polyacrylates or polymethacrylates with partially free functional groups; and/or, if required, effervescent agents, such as the above-mentioned starches, also carboxymethyl starch, cross-linked polyvinylpyrrolidone, agar, or alginic acid or a salt thereof, such as sodium alginate. Auxiliaries are primarily flow-regulating agents and lubricating agents, for example, silicic acid, talcum, stearic acid or salts thereof, such as magnesium stearate or calcium stearate. Dragee cores are provided with suitable coatings, optionally resistant to gastric juices, whereby there are used, inter alia, concentrated sugar solutions optionally containing gum arabic, talcum, polyvinylpyrrolidone, and/or titanium dioxide, lacquer solutions in aqueous solvents or, for producing coatings resistant to is stomach juices, solutions of esters of polyacrylates or polymethacrylates having partially free functional groups, or of suitable cellulose preparations such as acetylcellulose phthalate or hydroxypropylmethylcellulose phthalate, with or without suitable softeners such as phthalic acid ester or triacetin. Dyestuffs or pigments may be added to the tablets or dragee coatings, for example for identification or marking of the various doses of active ingredient. Formulation of pharmaceutical compositions is described further in U.S. Pat. No. 6,872,715 to Santi et al.

### EXAMPLE 1

Computer Modeling: Geldanamycin derivatives that have been assessed by molecular modeling studies to have a greater affinity toward hsp90 of Plasmodium falciparum and Trypanosoma Cruzi over that toward human hsp90 are the derivatives that possess one or more of the following modifications (see FIG. 1 for the numbering scheme of geldanamycin): (1) geldanamycin derivatives with a hydrogen-bonding atom as an oxygen (e.g., a hydroxyl group or ether group) or nitrogen (e.g., a primary, secondary or tertiary amine group) bonded to the methyl carbon atom on position 2 of geldanamycin; (2) geldanamycin derivatives with a hydrogen-bonding atom as an oxygen (e.g., a hydroxyl group or ether group) or nitrogen (e.g., a primary , secondary or tertiary amine group) bonded to the carbon atom of position 15 of geldanamycin (it is predicted that such a substituent will have increased affinity to the parasitic hsp90 when in the α-position on carbon 15); (3) geldanamycin derivatives in which the 7-position has been decarbamoylated and its free 7-hydroxyl group has been esterified with an α-aminoacetyl group or with an acyl group having a short alkyl or cycloalkyl attachment (thus an acyl group as propionyl, n-butyryl, α-methylpropionyl, and cyclopropylcarboxyl); and (4) geldanamycin derivatives in which the 11-hydroxyl group has been esterified with an acyl group having one or more positively charged groups, such as a positively charged nitrogen) on a γ- or ε-position (thus an acyl group as a γ-ammoniumbutyryl group or an α-(2-ammoniumethyl)-γ-ammoniumbutyryl group.

### EXAMPLE 2

General Methods. Melting points are uncorrected. Infrared spectra were recorded on a Matton Galaxy Series FTIR 3000 spectrophotometer. Ultraviolet-visible spectra were recorded on a Hitachi U-4001 spectrophotometer. ¹H and ¹³C NMR spectra were recorded on Varian Inova-600, UnityPlus-500, VRX-500 or VRX-300 spectrometers. The numbering used in all assignments is based on geldanamycin ring system unless otherwise indicated. Mass spectra were performed by the MSU Mass Spectrometry Facility. Anhydrous solvents were purified as standard methods.

Compound G: 17-(1-Azetidinyl)-17-demethoxygeldanamycin.

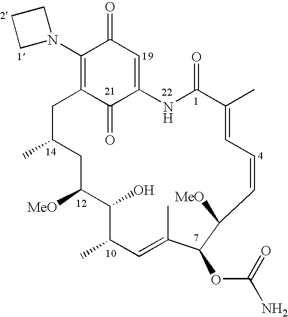

Azetidine (4.0 μl, 59 μmol) was added to a solution of (+)-geldanamycin (7.5 mg, 13 μmol) in dichloromethane (1.5 ml) with stirring. Upon the complete conversion of geldanamycin shown by thin layer chromatography (40 minutes), the mixture was washed with brine, dried over anhydrous sodium sulfate, and concentrated. Separation by flash column chromatography on silica gel (1:2 hexane/ethyl acetate) gave the product as a deep purple solid (7.7 mg, 98%). IR (in CH₂Cl₂) (cm⁻¹) 3422, 3075, 3049, 2986, 1733, 1686, 1651, 1605, 1540, 1486, 1420, 1375, 1283, 1260, 1103, 1047; ¹H NMR (CDCl₃, 500 MHz) δ 9.16 (s, 1H), 7.10 (s, 1H), 6.92 (bd, J=11.5 Hz, 1H), 6.56 (bdd, J=11.5, 11.0 Hz, 1H), 5.92 (bd, J=9.5 Hz, 1H), 5.82 (dd, J=11.0, 10.0 Hz, 1H), 5.15 (s, 1H), 4.79 (bs, 2H), 4.72-4.58 (m, 4H), 4.28 (bd, J=10.0 Hz, 1H), 3.54 (bd, J=9.0 Hz, 1H), 3.43 (ddd, J=9.0, 3.0, 3.0 Hz, 1H), 3.34 (s, 3H), 3.24 (s, 3H), 2.71 (dqd, J=9.5, 7.0, 2.0 Hz, 1H), 2.59 (d, J=14.0 Hz, 1H), 2.42 (quintet, J=8.0 Hz, 2H), 2.23 (dd, J=14.0, 11.0 Hz, 1H), 2.00 (bs, 3H), 1.78 (bs, 3H), 1.77-1.73 (m, 2H), 1.69-1.62 (m, 1H), 0.97 (d, J=7.0 Hz, 3H), 0.94 (d, J=6.5 Hz, 3H); ¹³C NMR (CDCl₃, 125 MHz) δ 185.8, 178.4, 168.4, 156.0, 145.9, 140.5, 135.5, 135.1, 134.0, 132.6, 126.7, 126.6, 109.6, 109.2, 81.8, 81.6, 81.3, 72.5, 58.9, 57.1, 56.7, 35.1, 34.1, 32.3, 28.1, 22.9, 18.4, 12.7, 12.6, 12.2; MS (FAB) found 586 [M+H]⁺.

Compound D: 17-(1-Azetidinyl)-7-decarbamyl-17-demethoxygeldanamycin.

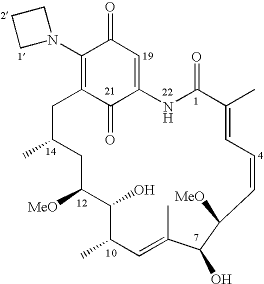

17-(1-Azetidinyl)-7-decarbamyl-17-demethoxy-geldanamycin has been synthesized previously by Schnur and Corman, J. Org. Chem. 59(9): p. 2583 (1994). Our procedure is done in a different solvent with higher yield. Potassium tert-butoxide (5.3 mg, 45 μmol) was added to a solution of 17-(1-azetidinyl)-17-demethoxygeldanamycin (5.0 mg, 8.5 μmol) in tert-butanol (4.0 ml) under nitrogen atmosphere. The reaction was stirred at room temperature for 1 hour, and then quenched by partitioning between ethyl acetate and brine. The organic layer was washed with brine, dried over anhydrous sodium sulfate, and concentrated. Separation by flash column chromatography on silica gel (1:2 hexane/ethyl acetate) gave the product as a purple solid (4.4 mg, 95%). IR (KBr) (cm⁻¹) 3461, 3330, 2955, 2927, 2871, 2826, 1685, 1652, 1539, 1489, 1404, 1381, 1287, 1255, 1191, 1136, 1106; ¹H NMR (CDCl₃, 500 MHz) δ 9.16 (s, 1H), 7.09 (s, 1H), 6.90 (bd, J=11.5 Hz, 1H), 6.54 (bdd, J=11.5, 11.0 Hz, 1H), 5.98 (dd, J=11.0, 10.0 Hz, 1H), 5.70 (bd, J=9.5 Hz, 1H), 4.72-4.59 (m, 4H), 4.16 (bd, J=10.0 Hz, 1H), 3.98 (s, 1H), 3.52 (dd, J=9.0, 2.0 Hz, 1H), 3.41 (ddd, J=9.0, 3.0, 3.0 Hz, 1H), 3.34 (s, 3H), 3.23 (s, 3H), 2.73 (dqd, J=9.5, 7.0, 2.0 Hz, 1H), 2.57 (d, J=14.0 Hz, 1H), 2.42 (quintet, J=8.0 Hz, 2H), 2.23 (dd, J=14.0, 11.0 Hz, 1H), 2.01 (d, J=1.0 Hz, 3H), 1.77-1.71 (m, 2H), 1.74 (d, J=1.0 Hz, 3H), 1.70-1.62 (m, 1H), 0.97 (d, J=7.0 Hz, 3H), 0.94 (d, J=6.5 Hz, 3H); ¹³C NMR (CDCl₃, 125 MHz) γ 185.8, 178.4, 168.6, 145.8, 140.5, 137.2, 136.1, 134.8, 132.0, 126.9, 125.9, 109.5, 109.2, 81.8, 80.5, 80.3, 72.9, 58.9, 56.7, 56.3, 34.9, 34.2, 32.2, 28.2, 22.9, 18.4, 12.6, 12.4, 11.8; MS (FAB) found 543 [M+H]⁺.

Compound F: 17-(1-Azetidinyl)-7-decarbamyl-17-demethoxy-7-propionylgeldanamycin.

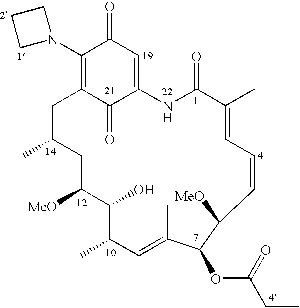

Propionic anhydride (5.0 μl, 39 μmol) was added to a solution of 17-(1-azetidinyl)-7-decarbamyl-17-demethoxygeldanamycin (2.0 mg, 3.7 μmol) and DMAP (5.0 mg, 41 μmol) in dichloromethane (0.8 ml) at room temperature with stirring. Upon the complete conversion of the starting material shown by thin layer chromatography (20 hours), the mixture was separated by flash column chromatography on silica gel (1:1 hexane/ethyl acetate) to afford a purple solid (1.7 mg, 77%). IR (KBr) (cm⁻¹) 3440, 3332, 2929, 2821, 1738, 1684, 1651, 1533, 1487, 1406, 1381, 1288, 1186, 1103; ¹H NMR (CDCl₃, 500 MHz) δ 9.18 (s, 1H), 7.09 (s, 1H), 6.94 (bd, J=11.5 Hz, 1H), 6.53 (bdd, J=11.5, 11.0 Hz, 1H), 5.88 (bd, J=9.5 Hz, 1H), 5.74 (dd, J=11.0, 10.0 Hz, 1H), 5.25 (s, 1H), 4.73-4.58 (m, 4H), 4.29 (bd, J=10.0 Hz, 1H), 3.55 (dd, J=9.0, 2.0 Hz, 1H), 3.43 (ddd, J=9.0, 3.0, 3.0 Hz, 1H), 3.34 (s, 3H), 3.23 (s, 3H), 2.70 (dqd, J=9.5, 7.0, 2.0 Hz, 1H), 2.60 (d, J=14.0 Hz, 1H), 2.45-2.39 (m, 4H), 2.23 (dd, J=14.0, 11.0 Hz, 1H), 2.00 (bs, 3H), 1.75 (d, J=1.0 Hz, 3H), 1.75-1.52 (m, 3H), 1.15 (t, J=7.6 Hz, 3H), 0.97 (d, J=7.0 Hz, 3H), 0.95 (d, J=6.5 Hz, 3H); ¹³C NMR (CDCl₃, 125 MHz) δ 185.8, 178.6, 173.7, 168.4, 146.0, 140.5, 135.7, 135.0, 133.8, 132.6, 126.8, 126.4, 109.6, 109.2, 81.7, 81.2, 80.7, 72.6, 58.8, 57.1, 56.7, 35.2, 34.0, 32.3, 28.3, 27.6, 22.8, 18.4, 12.7, 12.5, 12.3, 9.2; HRMS (FAB) found 599.3329 [M+H]⁺. calcd. 599.3333 for C₃₃H₄₇N₂O₈.

Compound B: 17-(1-Azetidinyl)-7-butyryl-7-decarbamyl-17-demethoxygeldanamycin.

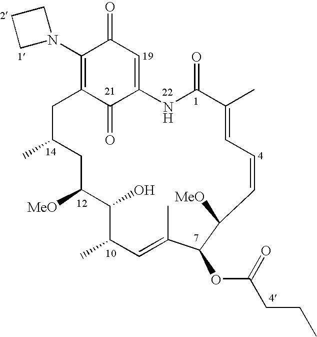

Butyric anhydride (3.7 μl, 22 μmol) was added to a solution of 17-(1-azetidinyl)-7-decarbamyl-17-demethoxygeldanamycin (2.0 mg, 3.7, μmol) and DMAP (3.5 mg, 29 μmol) in dichloromethane (1.0 ml) at room temperature with stirring. Upon the complete conversion of the starting material shown by thin layer chromatography (20 hours), the mixture was separated by flash column chromatography on silica gel (1:1 hexane/ethyl acetate) to afford a purple solid (2.1 mg, 95%). IR (KBr) (cm⁻¹) 3441, 3337, 2963, 2930, 1735, 1684, 1653, 1534, 1486, 1407, 1381, 1288, 1254, 1186, 1102; ¹H NMR (CDCl₃, 500 MHz) δ 9.18 (s, 1H), 7.10 (s, 1H), 6.93 (bd, J=11.5 Hz, 1H), 6.53 (ddd, J=11.5, 11.0, 1.0 Hz, 1H), 5.88 (bd, J=9.5 Hz, 1H), 5.74 (dd, J=11.0, 10.0 Hz, 1H), 5.26 (s, 1H), 4.73-4.58 (m, 4H), 4.29 (d, J=10.0 Hz, 1H), 3.54 (ddd, J=9.0, 6.5, 2.0 Hz, 1H), 3.43 (ddd, J=9.0, 3.0, 3.0 Hz, 1H), 3.34 (s, 3H), 3.23 (s, 3H), 2.70 (dqd, J=9.5, 7.0, 2.0 Hz, 1H), 2.60 (d, J=14.0 Hz, 1H), 2.42 (quintet, J=8.0 Hz, 2H), 2.38 (t, J=7.5 Hz, 2H), 2.22 (dd, J=14.0, 11.0 Hz, 1H), 2.00 (bs, 3H), 1.78-1.62 (m, 5H), 1.75 (d, J=1.0 Hz, 3H), 0.96 (d, J=7.0 Hz, 3H), 0.95 (d, J=6.5 Hz, 3H), 0.93 (t, J=7.5 Hz, 3H); HRMS (FAB) found 612.3412 [M]⁺. calcd. 612.3411 for C₃₄H₄₈N₂O₈.

17-(1-Azetidinyl)-7-decarbamyl-17-demethoxy-7-isobutyryl-geldanamycin.

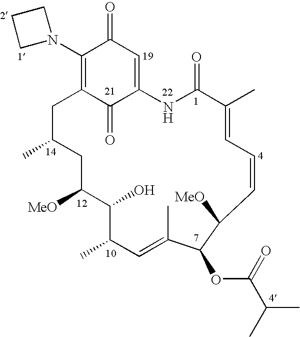

Isobutyric anhydride (5.0 μl, 30 μmol) was added to a solution of 17-(1-azetidinyl)-7-decarbamyl-17- demethoxygeldanamycin (1.7 mg, 3.1 μmol) and DMAP (4.8 mg, 39 μmol) in dichloromethane (0.8 ml) at room temperature with stirring. Upon the complete conversion of the starting material shown by thin layer chromatography (20 hours), the mixture was separated by flash column chromatography on silica gel (1:1 hexane/ethyl acetate) to give the product as a purple solid (1.8 mg, 96%). IR (KBr) (cm⁻¹) 3455, 3332, 2925, 2823, 1736, 1684, 1651, 1533, 1487, 1406, 1383, 1288, 1255, 1188, 1103; ¹H NMR (CDCl₃, 500 MHz) δ 9.19 (s, 1H), 7.09 (s, 1H), 6.95 (bd, J=11.5 Hz, 1H), 6.53 (ddd, J=11.5, 11.0, 1.0 Hz, 1H), 5.87 (bd, J=9.5 Hz, 1H), 5.73 (dd, J=11.0, 10.0 Hz, 1H), 5.23 (s, 1H), 4.73-4.58 (m, 4H), 4.29 (d, J=10.0 Hz, 1H), 3.55 (ddd, J=9.0, 6.5, 2.0 Hz, 1H), 3.43 (ddd, J=9.0, 3.0, 3.0 Hz, 1H), 3.34 (s, 3H), 3.23 (s, 3H), 2.74-2.64 (m, 2H), 2.61 (d, J=14.0 Hz, 1H), 2.42 (quintet, J=8.0 Hz, 2H), 2.22 (dd, J=14.0, 11.0 Hz, 1H), 2.00 (bs, 3H), 1.80-1.64 (m, 3H), 1.75 (d, J=1.0 Hz, 3H), 1.20-1.17 (m, 6H), 0.97 (d, J=7.0 Hz, 3H), 0.95 (d, J=6.5 Hz, 3H); HRMS (FAB) found 612.3414 [M]⁺. calcd. 612.3411 for C₃₄H₄₈N₂O₈.

17-(1-azetidinyl)-7-decarbamyl-7,11-diisobutyryl-17-demethoxygeldanamycin.

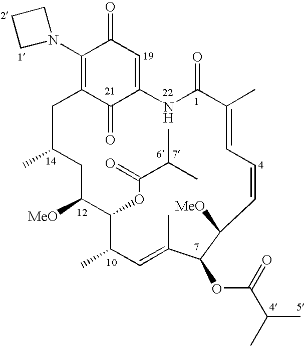

Isobutyric anhydride (5.0 μl, 30 μmol) was added to a solution of 17-(1-azetidinyl)-7-decarbamyl-17-demethoxygeldanamycin (1.0 mg, 1.8 μmol) and DMAP (4.7 mg, 34 μmol) in dichloromethane (0.5 ml) at room temperature with stirring. Upon the complete conversion of the starting material shown by thin layer chromatography (24 hours), the mixture was separated by flash column chromatography on silica gel (2:1 hexane/ethyl acetate) to give the product as a purple solid (1.1 mg, 87%). IR (KBr) (cm⁻¹) 3311, 2968, 2931, 2876, 2826, 1734, 1693, 1651, 1550, 1487, 1382, 1297, 1257, 1191, 1154, 1101; ¹H NMR (CDCl₃, 500 MHz) δ 9.45 (s, 1H), 7.05 (bd, J=11.5 Hz, 1H), 6.93 (s, 1H), 6.42 (ddd, J=11.5, 11.0, 1.0 Hz, 1H), 5.76 (dd, J=11.0, 7.0 Hz, 1H), 5.68 (bs, 1H), 5.24 (d, J=10.0, 1H), 5.03 (dd, J=9.0, 3.0 Hz, 1H), 4.65-4.53 (m, 4H), 4.50 (bd, J=7.0 Hz, 1H), 3.66 (bd, J=6.0 Hz, 1H), 3.32 (s, 3H), 3.29 (s, 3H), 2.82-2.72 (m, 2H), 2.68 (septet, J=7.0 Hz, 1H), 2.51 (septet, J=7.0, 1H), 2.38 (quintet, J=8.0 Hz, 2H), 1.99-1.93 (m, 1H), 1.97 (bs, 3H), 1.68-1.58 (m, 2H), 1.66 (bs, 3H), 1.25-1.29 (m, 1H), 1.17 (d, J=7.0, 6H), 1.14-1.11 (m, 6H), 0.96 (d, J=7.0 Hz, 3H), 0.92 (d, J=6.5 Hz, 3H); MS (FAB) found 683 [M+H]⁺.

Compound C: 17-(1-Azetidinyl)-7-(cyclopropanyl)carbonyl-7-decarbamyl-17-demethoxygeldanamycin.

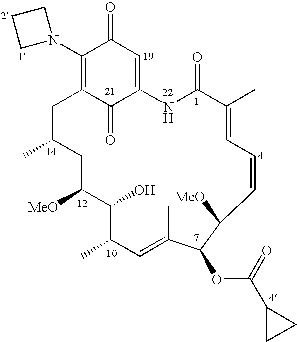

Under nitrogen protection, cyclopropane carboxylic acid (78 μl, 0.99 mmol) was added to a solution of DCC (102 mg, 0.49 mmol) in dichloromethane (1.0 ml) at room temperature. After 3 hours, the resulted white suspension was filtered, and the clear solution stirred with 17-(1-azetidinyl)-7-decarbamyl-17-demethoxygeldanamycin (1.7 mg, 3.1 μmol) and DMAP (10.0 mg, 82 μmol). Upon the complete conversion of the starting material shown by thin layer chromatography (3 hours), the mixture was separated by flash column chromatography on silica gel (1:1 hexane/ethyl acetate) to afford the product as a purple solid (1.7 mg, 89%). IR (KBr) (cm⁻¹) 3395, 3333, 2921, 2850, 1729, 1685, 1647, 1534, 1487, 1383, 1288, 1258, 1173, 1103; ¹H NMR (CDCl₃, 500 MHz) δ 9.18 (s, 1H), 7.09 (s, 1H), 6.94 (bd, J=11.5 Hz, 1H), 6.54 (ddd, J=11.5, 11.0, 1.0 Hz, 1H), 5.86 (bd, J=9.5 Hz, 1H), 5.77 (dd, J=11.0, 10.0 Hz, 1H), 5.25 (s, 1H), 4.73-4.58 (m, 4H), 4.28 (d, J=10.0 Hz, 1H), 3.54 (ddd, J=9.0, 6.5, 2.0 Hz, 1H), 3.43 (ddd, J=9.0, 3.0, 3.0 Hz, 1H), 3.34 (s, 3H), 3.24 (s, 3H), 2.71 (dqd, J=9.5, 7.0, 2.0 Hz, 1H), 2.60 (d, J=14.0 Hz, 1H), 2.42 (quintet, J=8.0 Hz, 2H), 2.22 (dd, J=14.0, 11.0 Hz, 1H), 2.00 (bs, 3H), 1.79-1.56 (m, 4H), 1.78 (d, J=1.0 Hz, 3H), 1.03-0.99 (m, 2H), 0.97 (d, J=7.0 Hz, 3H), 0.95 (d, J=6.5 Hz, 3H), 0.88-0.84 (m, 2H); HRMS (FAB) found 611.3336 [M+H]⁺. calcd. 611.3333 for C₃₄H₄₇N₂O₈.

Compound E: 17-(1-Azetidinyl)-7-benzoyl-7-decarbamyl-17-demethoxygeldanamycin.

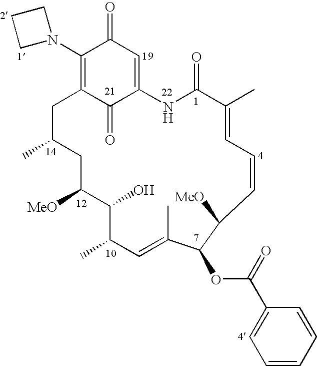

Benzoyl chloride (6.0 μl, 52 μmol) was added to a solution of 17-(1-azetidinyl)-7-decarbamyl-17-demethoxygeldanamycin (2.0 mg, 3.7 μmol) and DMAP (8.2 mg, 67 μmol) in dichloromethane (1.0 ml) at room temperature with stirring. Upon the complete conversion of the starting material shown by thin layer chromatography (3 hours), the mixture was taken up with EtOAc, washed with brine, dried over sodium sulfate, and concentrated. Separation by flash column chromatography on silica gel (1:1 hexane/ethyl acetate) afforded the product as a purple solid (3.2 mg, 82%).IR (KBr) (cm⁻¹) 3432, 3336, 2927, 2823, 1722, 1685, 1652, 1534, 1486, 1406, 1380, 1266, 1188, 1102; ¹H NMR (CDCl₃, 500 MHz) δ 9.21 (s, 1H), 8.12 (d, J=7.5 Hz, 1H), 7.57-7.53 (m, 2H), 7.46-7.42 (m, 2H), 7.11 (s, 1H), 6.99 (bd, J=11.5 Hz, 1H), 6.52 (ddd, J=11.5, 11.0, 1.0 Hz, 1H), 5.97 (bd, J=9.5 Hz, 1H), 5.80 (dd, J=11.0, 10.0 Hz, 1H), 5.47 (s, 1H), 4.77 (bs, 1H), 4.77-4.62 (m, 4H), 4.39 (d, J=10.0 Hz, 1H), 3.57 (ddd, J=9.0, 6.5, 2.0 Hz, 1H), 3.44 (ddd, J=9.0, 3.0, 3.0 Hz, 1H), 3.35 (s, 3H), 3.26 (s, 3H), 2.73 (dqd, J=9.5, 7.0, 2.0 Hz, 1H), 2.62 (d, J=14.0 Hz, 1H), 2.43 (quintet, J=8.0 Hz, 2H), 2.24 (dd, J=14.0, 11.0 Hz, 1H), 1.99 (bs, 3H), 1.84 (d, J=1.0 Hz, 3H), 1.78-1.73 (m, 2H), 1.73-1.66 (m, 1H), 0.97 (d, J=7.0 Hz, 3H), 0.95 (d, J=6.5 Hz, 3H); ¹³C NMR (CDCl₃, 125 MHz) δ 185.8, 178.5, 168.5, 165.8, 145.9, 140.5, 136.0, 135.0, 133.9, 132.9, 132.7, 130.4, 129.9, 128.4, 126.9, 126.4, 109.6, 109.2, 81.6, 81.4, 72.5, 58.9, 57.0, 56.7, 35.1, 34.1, 32.3, 28.2, 22.9, 18.5, 12.9, 12.6, 12.3; HRMS (FAB) found 647.3335 [M+H]⁺. calcd. 647.3333 for C₃₇H₄₇N₂O₈.

17-(1-Azetidinyl)-11-O-[4-(Boc)aminobutyryl]-17-demethoxygeldanamycin.

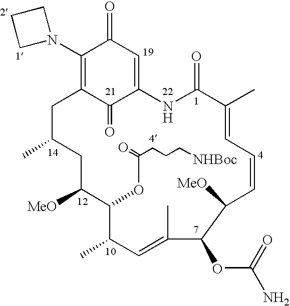

Under nitrogen protection, 2,4,6-trichlorobenzoyl chloride (4.9 μl, 30 μmol) was added to a solution of triethylamine (5.0 μl, 36 μmol) and 4-(Boc)aminobutyric acid (6.1 mg, 30 μmol) in dry THF (1.0 ml) at room temperature with stirring. After 4 hours, the resulted white suspension was filtered, and the clear solution stirred with 17-(1-azetidinyl)-17-demethoxygeldanamycin (3.2 mg, 5.5 μmol) and DMAP (3.7 mg, 30 μmol). Upon the complete conversion of the starting material shown by thin layer chromatography (18 hours), the mixture was taken up with EtOAc, washed with brine, dried over sodium sulfate, and concentrated. Separation by flash column chromatography on silica gel (1:1 hexane/ethyl acetate) afforded the product as a purple solid (3.8 mg, 90%). IR (KBr) (cm⁻¹) 3442, 3363, 2954, 2930, 1730, 1698, 1651, 1601, 1549, 1487, 1377, 1286, 1256; ¹H NMR (CDCl₃, 500 MHz) δ 9.41 (s, 1H), 9.37 (s, 1H), 7.09 (bs, 1H), 6.94 (s, 1H), 6.49 (ddd, J=11.5, 11.0, 1.0 Hz, 1H), 5.82 (dd, J=11.0, 7.5 Hz, 1H), 5.50 (bs, 1H), 5.31 (bs, 1H), 5.08 (bd, J=6.5 Hz, 1H), 4.76 (bs, 1H), 4.65-4.53 (m, 4H), 4.48 (bs, 1H), 3.65 (bs, 1H), 3.33 (s, 3H), 3.30 (s, 3H), 3.16-3.06 (m, 2H), 2.85-2.72 (m, 2H), 2.38 (quintet, J=8.0 Hz, 2H), 2.30 (t, J=7.5 Hz, 2H), 2.03-1.98 (m, 1H), 1.98 (bs, 3H), 1.74 (tt, J=7.5, 7.5 Hz, 2H), 1.67 (bs, 3H), 1.65-1.55 (m, 2H), 1.41 (s, 9H), 1.32-1.20 (m, 1H), 0.96 (d, J=7.0 Hz, 3H), 0.93 (d, J=6.5 Hz, 3H); HRMS (FAB) found 771.4182 [M+H]⁺. calcd. 771.4180 for C₄₀H₅₉N₄O₁₁.

Compound A: 11-O-(4-Aminobutyryl)-17-(1-azetidinyl)-17-demethoxygeldanamycin trifluoroacetate.

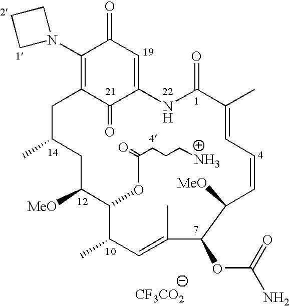

At room temperature, trifluoroacetic acid (100 μl) was added into a solution of 17-(1-azetidinyl)-11-O-[4-(Boc)aminobutyryl]-17-demethoxygeldanamycin (1.5 mg, 2.0 μmol) in dichloromethane (1.5 ml). Upon the complete conversion of the starting material shown by thin layer chromatography (1 hour), the solvent was removed to afford the product as a purple solid (1.5 mg, 97%). HRMS (FAB) found 785.3588 [M+H]⁺. calcd. 785.3585 for C₃₇H₅₂N₄O₁₁F₃.

### EXAMPLE 3

TABLE 1
Testing of Compounds A-G against P. falciparum.

| | Target | | | | |
|---|---|---|---|---|---|
| Compound | strain | IC50 | IC90 | [Start] | Units |
| Mefloquine | W2 | 1.629 | 4.0886 | 250 | ng/ml |
| Mefloquine | TM91C235 | 13.8151 | 29.1164 | 250 | ng/ml |
| Chloroquine | W2 | 29.6758 | 37.8321 | 1000 | ng/ml |
| Chloroquine | TM91C235 | 124.6805 | 159.8127 | 1000 | ng/ml |
| A | W2 | 649.5055 | 1787.864 | 10000 | ng/ml |
| A | TM91C235 | 1025.0364 | 2534.055 | 10000 | ng/ml |
| B | W2 | 785.512 | 2730.367 | 10000 | ng/ml |
| B | TM91C235 | 2713.8032 | 4019.62 | 10000 | ng/ml |
| C | W2 | 875.386 | 3063.072 | 10000 | ng/ml |
| C | TM91C235 | 2637.4199 | 3953.097 | 10000 | ng/ml |
| D | W2 | 957.6366 | 6176.345 | 10000 | ng/ml |
| D | TM91C235 | 4044.4473 | 8407.384 | 10000 | ng/ml |
| E | W2 | 1259.6951 | 5425.577 | 10000 | ng/ml |
| E | TM91C235 | 4288.0845 | 6066.255 | 10000 | ng/ml |
| F | W2 | 908.6389 | 3736.168 | 10000 | ng/ml |
| F | TM91C235 | 3034.5381 | 5543.161 | 10000 | ng/ml |
| G | W2 | 68.291 | 208.2086 | 10000 | ng/ml |
| G | TM91C235 | 169.2607 | 346.2624 | 10000 | ng/ml |

Table 1 shows the IC₅₀ and IC₉₀ values for compounds A through G when tested against two different P. falciparum target strains, W2 and TM91C235. Both parasite strains are chloroquine and pyrimethamine resistant and TM91C₂₃₅ is also mefloquine resistant.

### EXAMPLE 4

General procedure for reduction of derivatives of 17-amino-17-demethoxygeldanamycin. Procedure based on reduction of geldanamycin reported in: R. C. Schnur, et. Al., “Inhibition of the Oncogene Product p. 185^{erbB-2} in Vitro and in Vivo by Geldanamycin and Dihydrogeldanamycin Derivatives”, Journal of Medicinal Chemistry, volume 38, pages 3806-3812, year 1995.

The 17-amino-17-demethoxygeldanamycin derivative is dissolved in ethyl acetate (about 1 mmole of derivative to 50 milliliters ethyl acetate) and then an equivalent volume of an aqueous solution of 10% sodium dithionite is added. The mixture is stirred at room temperature for 0.5 to 2 hours. Under a nitrogen atmosphere, the organic layer is then separated, dried over an anhydrous solid drying such as magnesium sulfate, filtered and concentrated to give product (a 18,21-dihydro-17-amino-17-demethoxygeldanamycin derivative).

### EXAMPLE 5

General procedure for the protonation of the reduced 17-amino-17-demethoxygeldanamycin derivative. Procedure based on protonation of reduced 17-amino-17-demethoxygeldanamycin derivatives reported in: J. Ge, et. al., “Design, Synthesis, and biological Evaluation of Hydroquinone Derivatives of 17-Amino-17-demethoxygeldanamycin as Potent, Water-Soluble Inhibitors of Hsp90”, Journal of Medicinal Chemistry, volume 49, pages 4606-4615, year 2006.

The above 18,21-dihydro-17-amino-17-demethoxygeldanamycin derivative is dissolved in an aprotic organic solvent (such as ethyl acetate) and treated with an excess of a protic acid (such as 1-2 Molar hydrogen chloride in ethyl acetate). The protonated 18,21-dihydro-17-amino-17-demethoxygeldanamycin derivative produce precipitates out. Should the product not precipitate out, the solution can be concentrated and cooled to result in crystallization of the product. Choices of protic acid include, but are not limited to, hydrogen chloride, hydrogen bromide, hydrogen iodide, para-toluenesulfonic acid, benzenesulfonic acid, phosphoric acid, sodium dihydrogen phosphate, acetic acid, citric acid, benzoic acid, and para-toluic acid.

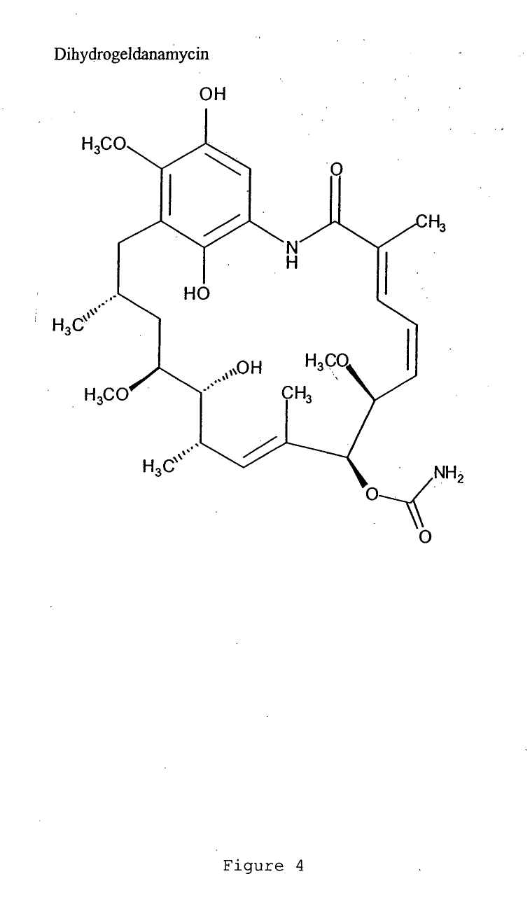

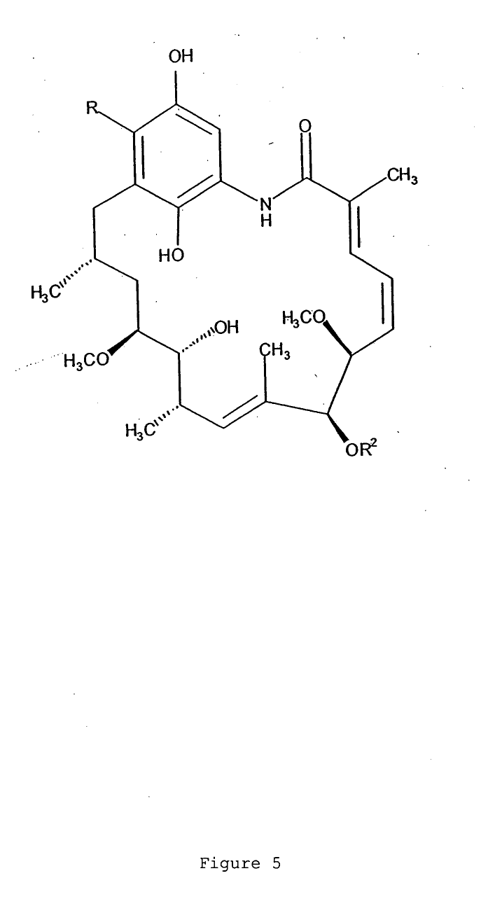

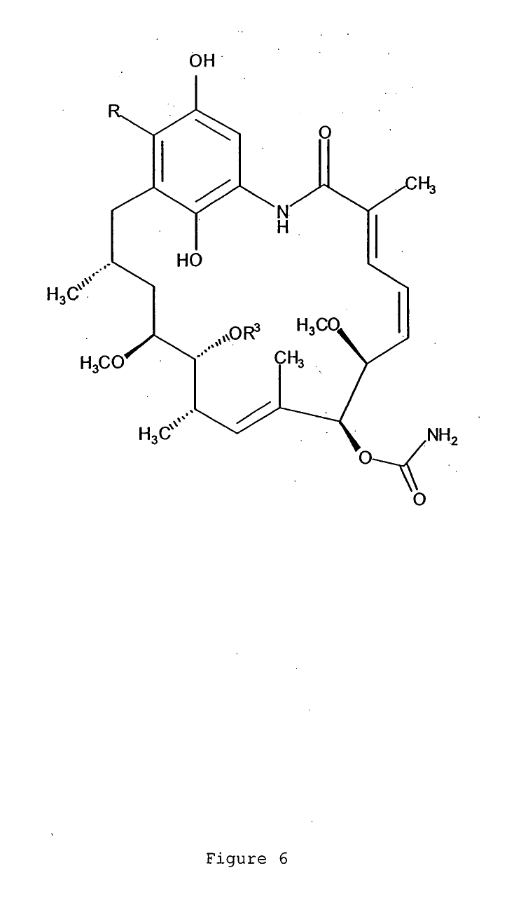

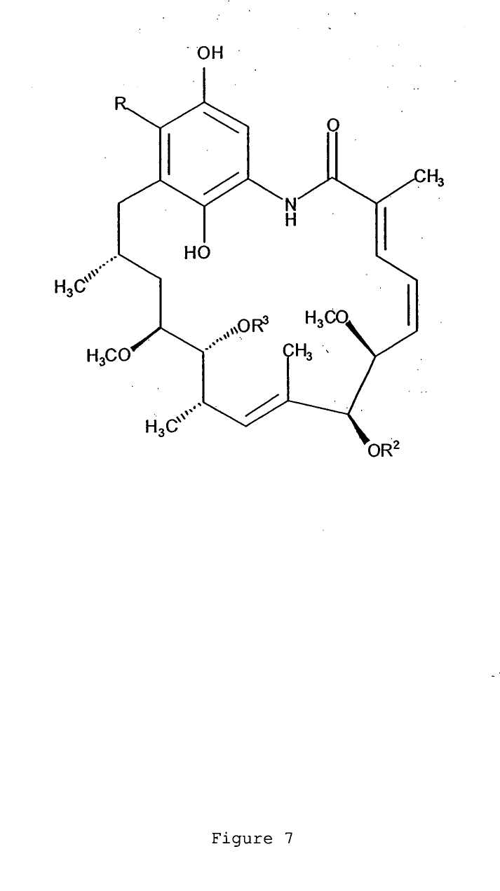

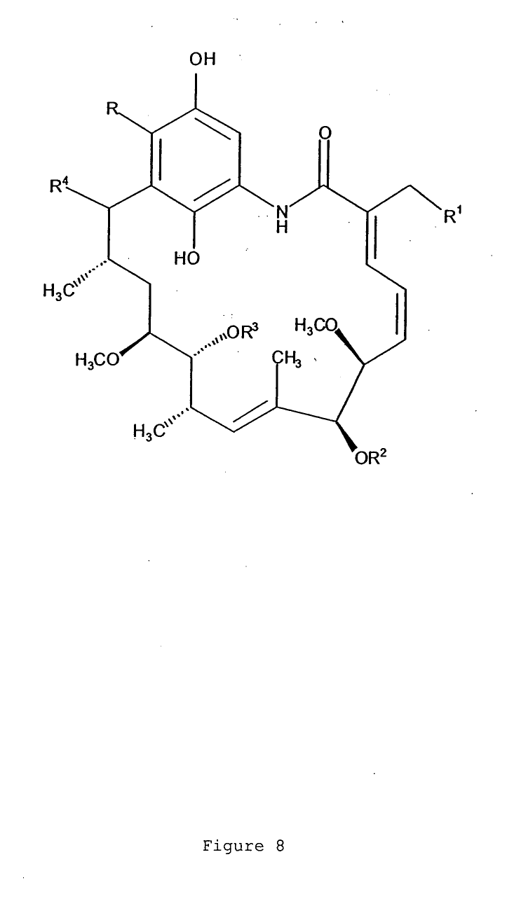

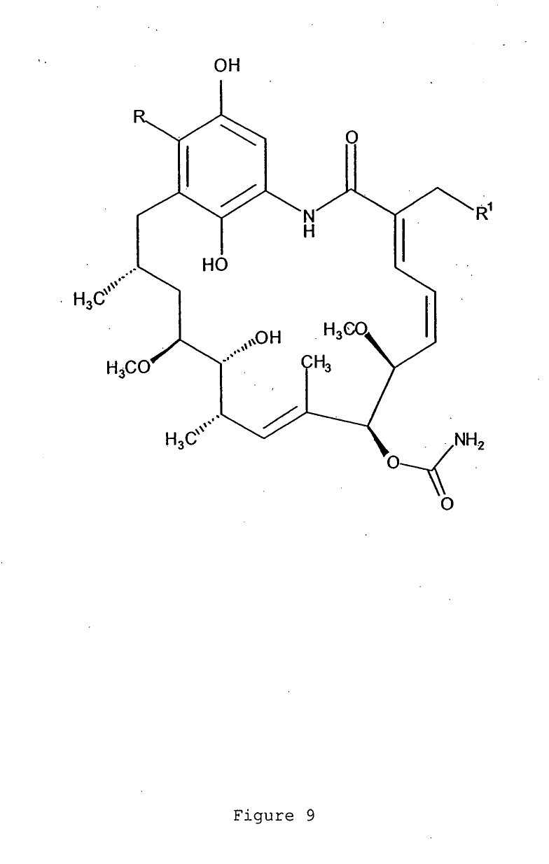

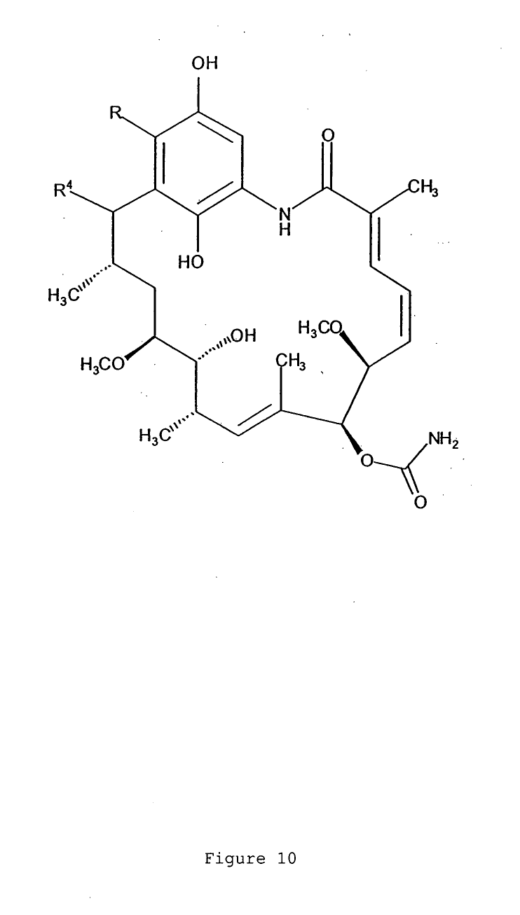

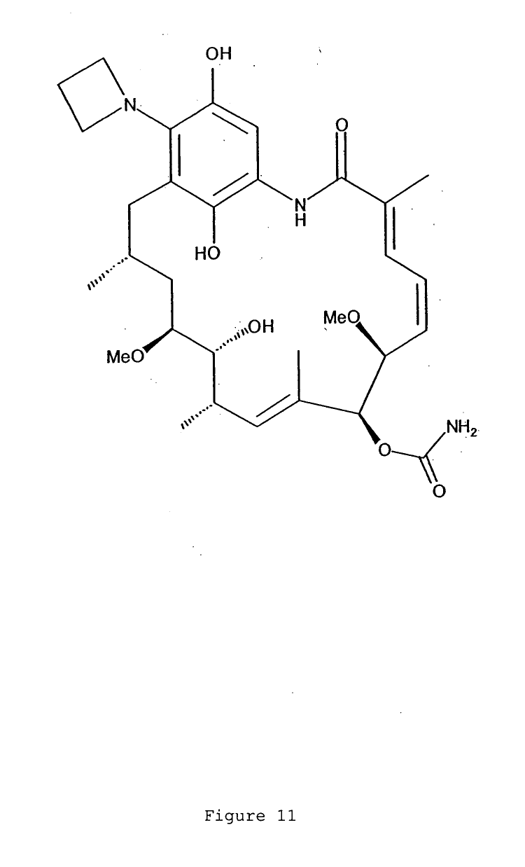

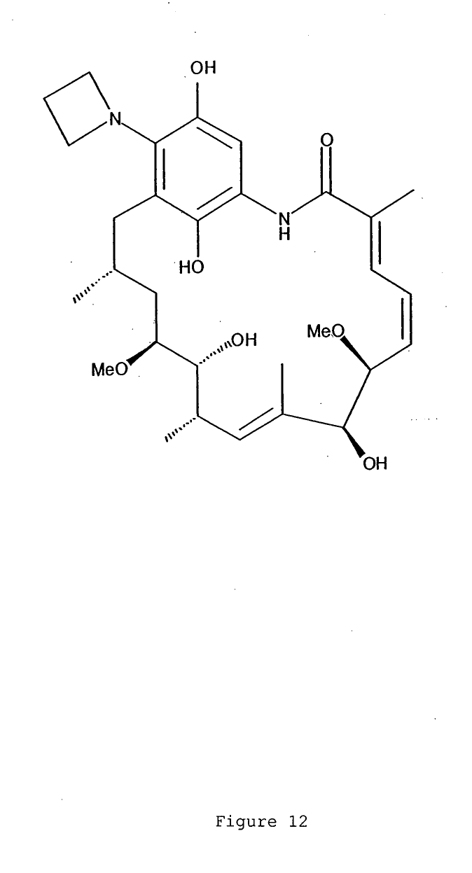

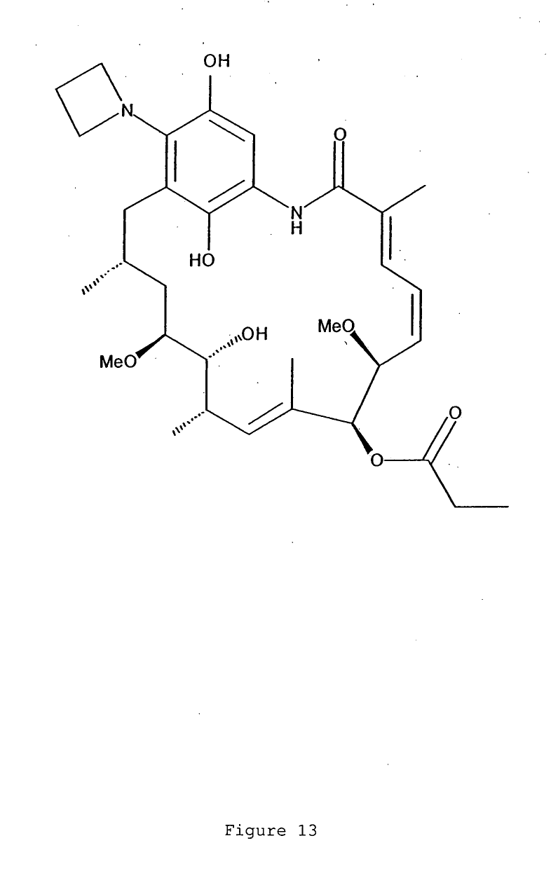

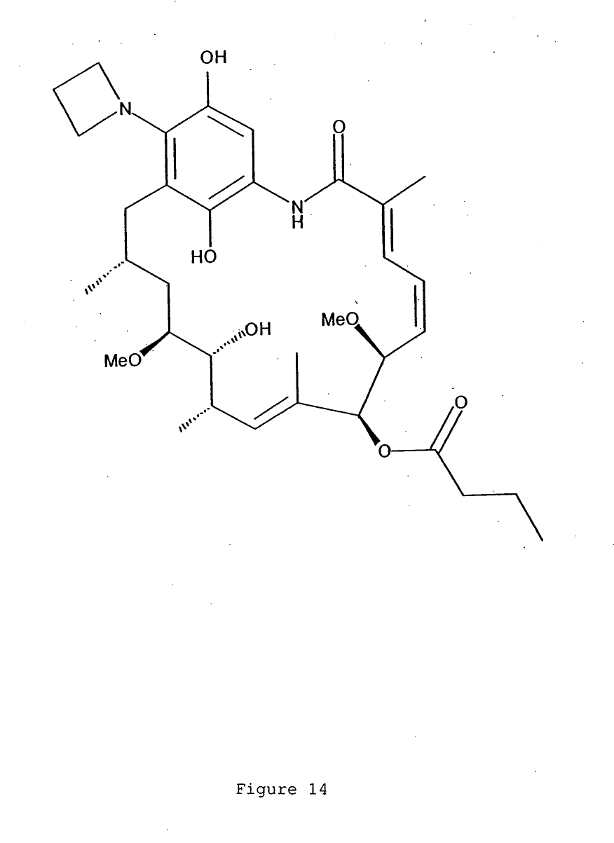

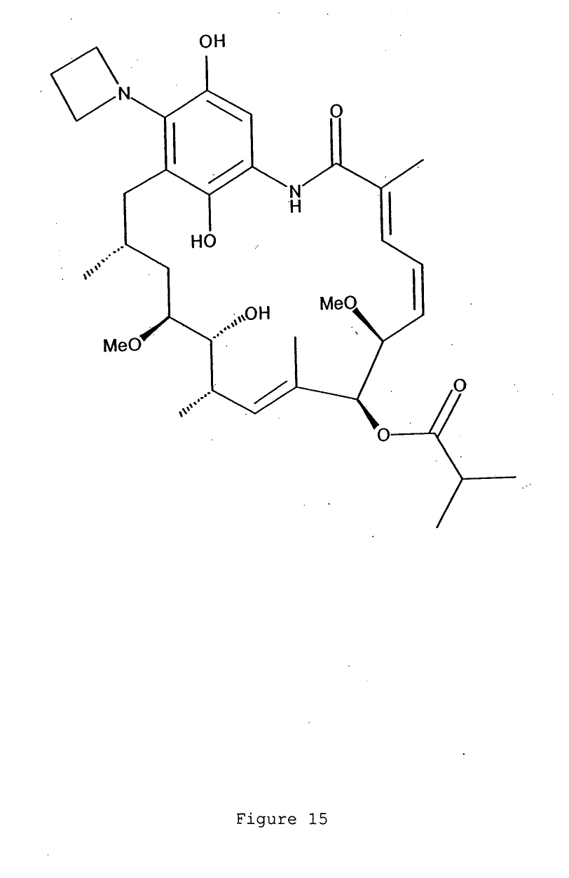

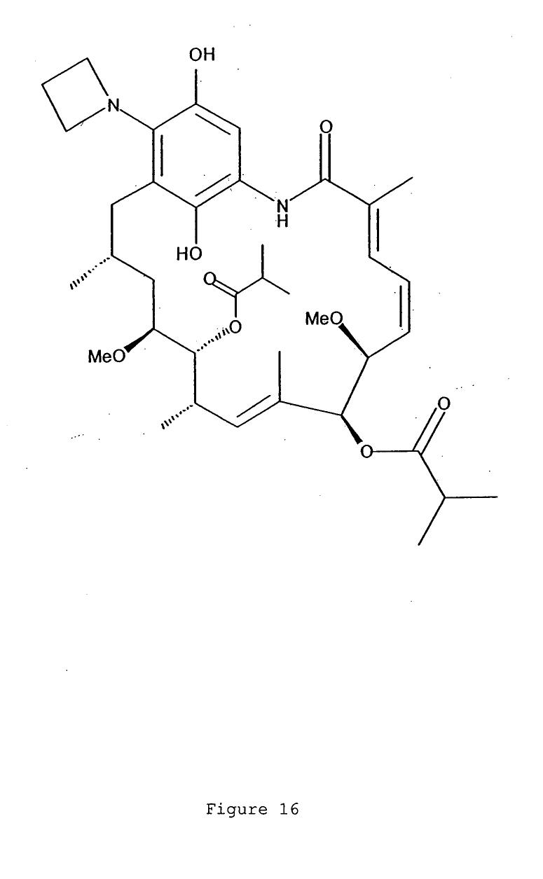

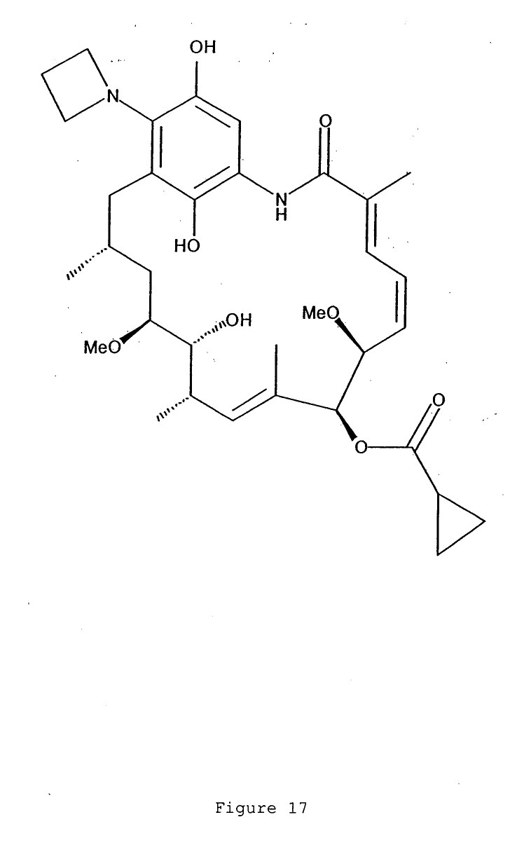

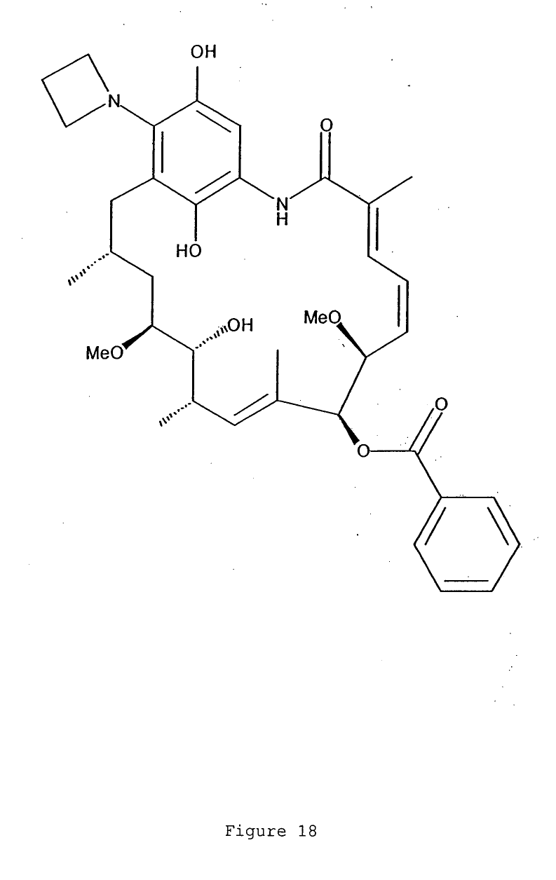

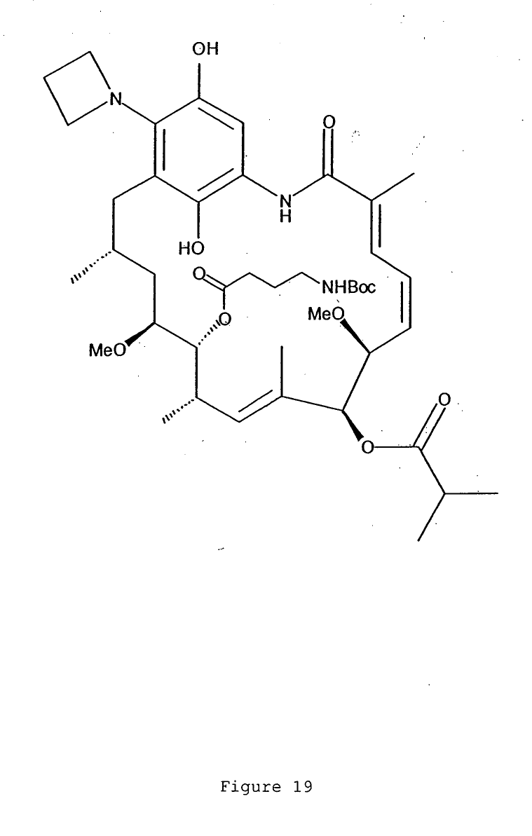

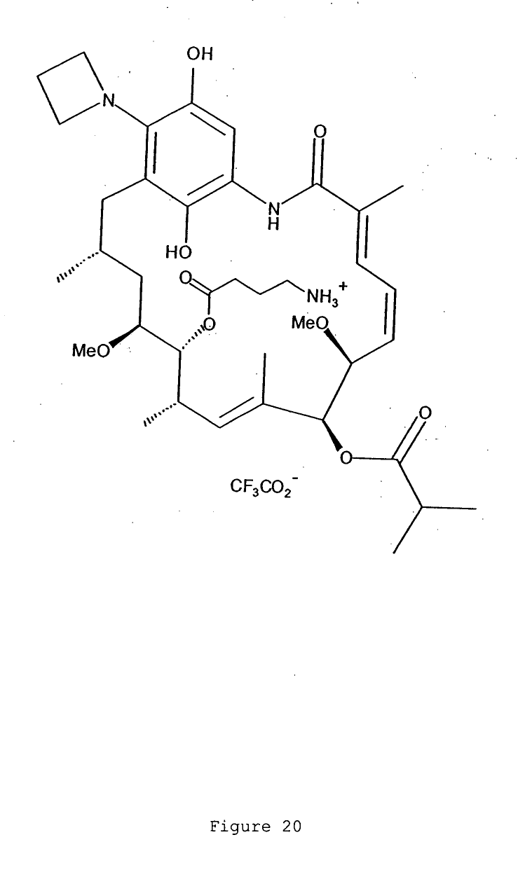

FIGS. 4 to 20 show the structures of compounds which are derived from compounds specifically described previously in the Examples. If there is a nitrogen in the 17 position, it is protonated in the said form.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A geldanamycin derivative with the structure: 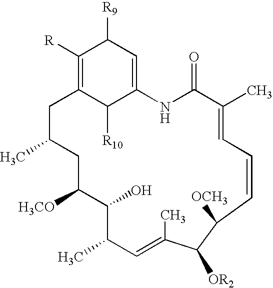
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocycle, aryl, or heteroaryl; or R⁵ and R⁶ and the nitrogen to which they are attached combine to form a substituted or unsubstituted 3, 4, 5, 6, or 7 membered ring;
wherein R² is an aminoacyl group with 1 to 6 carbon atoms, an acyl group with a phenyl moiety (i.e. a benzoyl group), or an acyl group with an alky or cycloalkyl moiety comprising 3 to 4 carbon atoms; and wherein R₉ and R₁₀are either hydroxy groups or keto groups an d amine or amino salts thereof.

2. The geldanamycin derivative of claim 1 wherein R² is a propionyl, n-butyryl, α-methylpropionyl, benzoyl, or cyclopropylcarboxyl group.

3. The geldanamycin derivative of claim 1 wherein R is an azetidinyl moiety.

4. The geldanamycin derivative of claim 1 wherein the derivative is 17-(1-azetidinyl)-7-butyryl-7-decarbamyl-17-demethoxygeldanamycin, 17-(1-azetidinyl)-7-(cyclopropanyl)-carbonyl-7-decarbamyl-17-demethoxygeldanamycin, 17-(1-azetidinyl)-7-benzoyl-7-decarbamyl-17-demethoxygeldanamycin, 17-(1-azetidinyl)-7-decarbamyl-17-demethoxy-7-propionyl-geldanamycin, or 17-(1-azetidinyl)-7-decarbamyl-17-demethoxy-7-isobutyryl-geldanamycin and hydroquinone derivatives thereof.

5. A geldanamycin derivative with the structure: 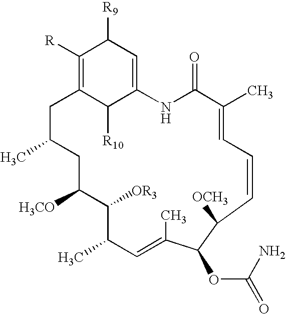
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring;
wherein R³ is an acyl group with 2 to 8 carbon atoms and one or more positively charged groups; and
wherein R₉ and R₁₀ and are either hydroxy groups or keto groups and amine or amino salts thereof.

6. The geldanamycin derivative of claim 5 wherein the positively charged group comprises nitrogen.

7. The geldanamycin derivative of claim 5 wherein R³ is a γ-ammonium butyryl acyl group or an α-(2-ammoniumethyl)-γ-ammonium butyryl acyl group.

8. The geldanamycin derivative of claim 5 wherein R is an azetidinyl moiety.

9. The geldanamycin derivative of claim 5 wherein the derivative is 11-O-(4-ammoniumbutyryl)-17-(1-azetidinyl)-17-demethoxygeldanamycin trifluoroacetate.

10. The geldanamycin derivative of claim 5 wherein the derivative is an 11-O-(4-ammoniumbutyryl)-17-(1-azetidinyl)-17-demethoxygeldanamycin salt having an acetate, fluoride, chloride, bromide, iodide, benzoate, phenylsulfonate, hydrogen sulfate or dihydrogen sulfate anion as a counterion.

11. A geldanamycin derivative having one or more substitutions, R, R², and R³, with the structure: 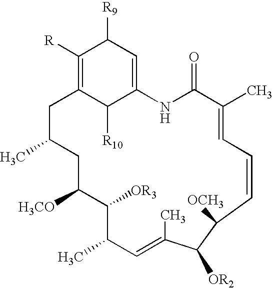
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring;
wherein R² and R³ are α-methylpropionyl groups; and
wherein R₉ and R₁₀ and are either hydroxy groups or keto groups and amine or amino salts thereof.

12. The geldanamycin derivative of claim 11 wherein the derivative is 17-(1-azetidinyl)-7-decarbamyl-7,11-diisobutyryl-17-demethoxygeldanamycin.

13. A geldanamycin derivative having one or more substitutions, R, R¹, R², R³ and R⁴, with the structure: 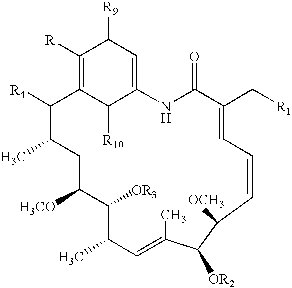
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and
wherein the one or more substitutions are organic groups selected from the groups consisting of:
(a) attached to the methylene carbon atom at position 2, R¹ is an organic group with 1 to 2 carbon atoms and a hydrogen-bonding atom replacing the hydrogen;
(b) at position 7, R² is an aminoacyl group with 1 to 6 carbon atoms, an acyl group with an alkyl moiety containing 3 to 4 carbon atoms, an acyl group with a phenyl moiety, or an acyl group with a cycloalkyl moiety containing 3 to 4 carbon atoms replacing the carbamoyl group;
(c) at position 11, R³ is an acyl group with 2 to 8 carbon atoms and one or more positively charged groups replacing the hydroxyl;
(d) at position 15, R⁴ is an a hydrogen-bonding atom replacing the hydrogen; and
(e) wherein R₉ and R₁₀ are either hydroxy groups or keto groups and amine or amino salts thereof.

14. The geldanamycin derivative of claim 13 wherein R is an azetidinyl moiety.

15. A geldanamycin derivative with the structure: 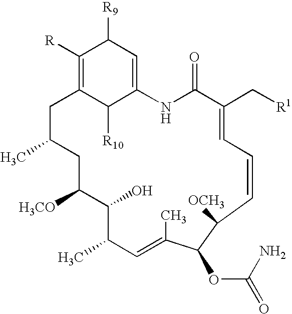
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring;
wherein R¹ is an organic group with 1 to 2 carbon atoms and with a hydrogen-bonding atom; and
wherein R₉ and R₁₀ and are either hydroxy groups or keto groups and amine or amino salts thereof.

16. The geldanamycin derivative of claim 15 wherein the hydrogen-bonding atom is an oxygen or a nitrogen atom.

17. The geldanamycin derivative of claim 15 wherein R¹ has a hydroxyl, ether, primary amine, secondary amine, or tertiary amine group and hydroquinone derivatives thereof.

18. A geldanamycin derivative with the structure: 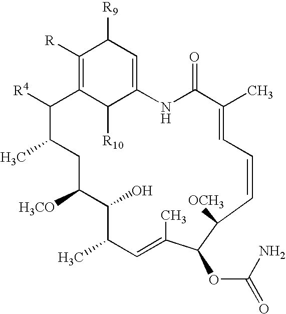
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring;
wherein R⁴ comprises a hydrogen-bonding atom which is bonded to the carbon atom; and
wherein R₉ and R₁₀ and are either hydroxy groups or keto groups and amine or amino salts thereof.

19. The geldanamycin derivative of claim 18 wherein the hydrogen-bonding atom is an oxygen or a nitrogen.

20. The geldanamycin derivative of claim 18 wherein R⁴ is a hydroxyl, alkoxy, primary amine, secondary amine, tertiary amine group, primary ammonium, secondary ammonium, or tertiary ammonium group.

21. A method of inhibiting Plasmodium falciparum comprising providing a geldanamycin derivative having one or more substitutions, R¹, R², R³ and R⁴, with the structure: 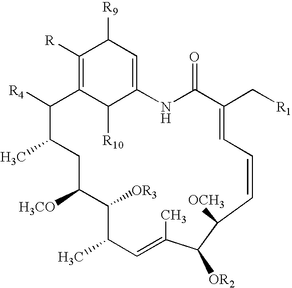
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and
wherein the one or more substitutions are organic groups selected from the groups consisting of:
(a) attached to the methylene carbon atom at position 2, R¹ is an organic group with 1 to 2 carbon atoms and a hydrogen-bonding atom replacing the hydrogen;
(b) at position 7, R² is an aminoacyl group with 1 to 6 carbon atoms, an acyl group with an alkyl moiety containing 3 to 4 carbon atoms, an acyl group with a phenyl moiety (i.e. a benzoyl group), or an acyl group with a cycloalkyl moiety containing 3 to 4 carbon atoms replacing the carbamoyl group;
(c) at position 11, R³ is an acyl group with 2 to 8 carbon atoms and one or more positively charged groups replacing the hydroxyl;
(d) at position 15, R⁴ is a hydrogen-bonding atom replacing the hydrogen; and
(e) wherein R₉ and R₁₀ and are either hydroxy groups or keto groups and amine or amino salts thereof.
so as to inhibit the Plasmodium falciparum.

22. The method of claim 21 wherein R is an azetidinyl moiety.

23. The method of claim 21 wherein R² is a propionyl, n-butyryl, α-methylpropionyl, benzoyl, or cyclopropylcarboxyl group.

24. The method of claim 23 wherein the derivative is 17-(1-azetidinyl)-7-butyryl-7-decarbamyl-17-demethoxygeldanamycin, 17-(1-azetidinyl)-7-(cyclopropanyl)-carbonyl-7-decarbamyl-17-demethoxygeldanamycin, 17-(1-azetidinyl)-7-benzoyl-7-decarbamyl-17-demethoxygeldanamycin, 17-(1-azetidinyl)-7-decarbamyl-17-demethoxy-7-propionyl-geldanamycin, or 17-(1-azetidinyl)-7-decarbamyl-17-demethoxy -7-isobutyryl-geldanamycin and hydroquinone derivatives thereof.

25. The method of claim 21 wherein the positively charged group comprises nitrogen.

26. The method of claim 25 wherein R³ is a γ-ammonium butyryl acyl group or an α-(2-ammoniumethyl)-γ-ammonium butyryl acyl group.

27. The method of claim 26 wherein the derivative is 11-O-(4-ammoniumbutyryl)-17-(1-azetidinyl)-17-demethoxygeldanamycin trifluoroacetate.

28. The method of claim 26 wherein the derivative is an 11-O-(4-ammoniumbutyryl)-17-(1-azetidinyl)-17-demethoxygeldanamycin salt having an acetate, fluoride, chloride, bromide, iodide, benzoate, phenylsulfonate, hydrogen sulfate or dihydrogen sulfate anion as a counterion.

29. A method of inhibiting a parasite heat shock protein homolog of human heat shock protein 90 (hsp90) comprising providing a geldanamycin derivative having one or more substitutions, R¹, R², R³ and R⁴, with the structure: 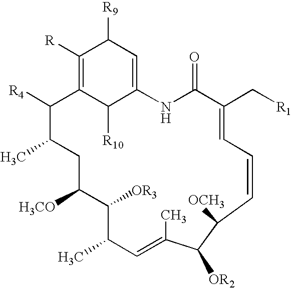
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and
wherein the one or more substitutions are organic groups selected from the groups consisting of:
(a) attached to the methylene carbon atom at position 2, R¹ is an organic group with 1 to 2 carbon atoms and a hydrogen-bonding atom replacing the hydrogen;
(b) at position 7, R² is an aminoacyl group with 1 to 6 carbon atoms, an acyl group with an alkyl moiety containing 3 to 4 carbon atoms, an acyl group with a phenyl moiety, or an acyl group with a cycloalkyl moiety containing 3 to 4 carbon atoms replacing the carbamoyl group;
(c) at position 11, R³ is an acyl group with 2 to 8 carbon atoms and one or more positively charged groups replacing the hydroxyl;
(d) at position 15, R⁴ is a hydrogen-bonding atom replacing the hydrogen; and
(e) wherein R₉ and R₁₀ are either hydroxy groups or keto groups and amine or amino salts thereof.
so as to inhibit the parasitic heat shock proteins.

30. The method of claim 29 wherein the parasite is selected from the group consisting of Plasmodium falciparum, Trypanosoma cruzi, and Leishmania donovani.

31. The method of claim 29 wherein the parasite is selected from the group consisting of protozoan, nematode, cestode and trematode parasites.

32. A method of treating a patient with a parasitic disease comprising providing to the patient a geldanamycin derivative having one or more substitutions, R¹, R², R³ and R⁴, with the structure: 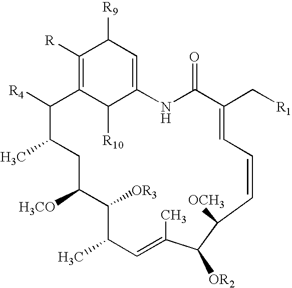
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; and
wherein the one or more substitutions are organic groups selected from the groups consisting of:
(a) attached to the methylene carbon atom at position 2, R¹ is an organic group with 1 to 2 carbon atoms and a hydrogen-bonding atom replacing the hydrogen;
(b) at position 7, R² is an aminoacyl group with 1 to 6 carbon atoms, an acyl group with an alkyl moiety containing 3 to 4 carbon atoms, an acyl group with a phenyl moiety, or an acyl group with a cycloalkyl moiety containing 3 to 4 carbon atoms replacing the carbamoyl group;
(c) at position 11, R³ is an acyl group with 2 to 8 carbon atoms and one or more positively charged groups replacing the hydroxyl;
(d) at position 15, R⁴ is a hydrogen-bonding atom replacing the hydrogen; and
(e) wherein R₉ and R₁₀ and are either hydroxy groups or keto groups and amine or amino salts thereof.
to inhibit a parasite heat shock protein homolog of human heat shock protein 90 (hsp90), so as to treat the disease.

33. The method of claim 32 wherein the parasite is selected from the group consisting of Plasmodium falciparum, Trypanosoma cruzi, and Leishmania donovani.

34. The method of claim 32 wherein the parasite is selected from the group consisting of protozoan, nematode, cestode and trematode parasites.

35. A method of treating a patient with cancer comprising providing to the patient a geldanamycin derivative having one or more substitutions, R, R², and R³, with the structure: 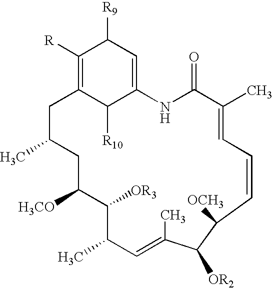
wherein R is methoxy or an R⁵R⁶N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring;
wherein R² is an aminoacyl group with 1 to 6 carbon atoms, an acyl group with a phenyl moiety, or an acyl group with an alkyl or cycloalkyl moiety comprising 3 to 4 carbon atoms;
wherein R³ is an acyl group with 2 to 8 carbon atoms and one or more positively charged groups; and
wherein R₉ and R₁₀ are either hydroxy groups or keto groups and amine or amino salts thereof
so as to treat the patient with cancer.

36. The geldanamycin derivative of claim 35 wherein R² is a propionyl, n-butyryl, α-methylpropionyl, benzoyl, or cyclopropylcarboxyl group.

37. The geldanamycin derivative of claim 35 wherein R³ is a γ-ammonium butyryl acyl group or an α-(2-ammoniumethyl)-γ-ammonium butyryl acyl group.

38. The geldanamycin derivative of claim 35 wherein R is an azetidinyl moiety.
